(19) United States
(12) Patent Application Publication
(10) Pub. No.: US 2026/0115205 A1
(43) Pub. Date: Apr. 30, 2026

(54) ON DEMAND FEMALE CONTRACEPTIVE

(71) Applicants: David Fitzgerald Archer, Norfolk, VA (US); Diane Marie Duffy, Norfolk, VA (US)

(72) Inventors: David Fitzgerald Archer, Norfolk, VA (US); Diane Marie Duffy, Norfolk, VA (US)

(21) Appl. No.: 18/287,294

(22) PCT Filed: Apr. 19, 2022
(86) PCT No.: PCT/US2022/025425

§ 371 Date: Oct. 18, 2023

Related U.S. Application Data
(60) Provisional application No. 63/176,763, filed on Apr. 19, 2021.

(51) Int. Cl. A61K 31/57; A61K 31/5415

(52) U.S. Cl. CPC A61K 31/57; A61K 31/5415

(57) ABSTRACT

The present invention provides methods of female contraception, the methods comprising administering an effective amount of a combination of a progesterone receptor agonist and a cyclooxygenase-2 inhibitor in each of two doses, the first dose is administered within about 24 hours before or after a first act of sexual intercourse and the second dose is administered between about 36 hours and about 60 hours after the first dose.

### FIELD OF THE INVENTION

The field of the invention generally relates to medicine and pharmaceuticals, in particular to methods of female contraception.

### BACKGROUND OF THE INVENTION

Fecundity is a term that embraces male and female reproductive events and outcomes, and this basic human drive has importance in continuing the species with pregnancy as the ultimate outcome. In modern societies, control over pregnancy and the number of children in a family is desired for many people in diverse cultures for multiple reasons. As countries and cultures have become more advanced, the need for large families has decreased. Population pressures from large families contribute to changes in the environment and increase the pressure on supplies of food and water. There are many male and female options that reduce fertility, are contraceptive and provide the family planning options that many couples seek. Each method has advantages and disadvantages, and women and men may change methods depending upon convenience and personal preferences.

Female fecundity requires ovulation, which is follicle rupture followed by a release of the oocyte (egg). The midcycle increase or surge of pituitary luteinizing hormone (LH) initiates a series of events within the ovarian follicle culminating in ovulation. The absence of a LH surge results in follicular atresia and anovulation. The normal midcycle surge of LH increases expression of ovarian intra-follicular prostaglandin-endoperoxide synthase 2 (PTGS2; also known as cyclooxygenase-2 (COX-2)), a key enzyme that synthesizes prostaglandin E2 (PGE2). PGE2 and its receptors induce protease activity in the follicle wall resulting in rupture, as well as expansion of the cumulus cells surrounding the oocyte, and the resumption of oocyte maturation. These events are highly synchronized and result in a mature oocyte being extruded at the time of follicle rupture. Oocyte survival in the fallopian tube is about 24 hours, with sperm survival in the female reproductive tract between four to five days. Hence, the four days prior to and including ovulation constitute the window of fertility, where intercourse results in 95% of all pregnancies. Intercourse on other cycle days results in very few pregnancies.

There are many contraceptive methods available to couples, the primary categories being hormonal and non-hormonal methods. There is a large difference between hormonal and non-hormonal contraceptives in terms of efficacy and safety. Most female hormonal contraceptives provide continuous exposure to exogenous hormones and by inhibiting ovulation are highly effective in preventing pregnancy. They are available as pills, transdermal delivery systems (patches), vaginal rings, injections, implants and intrauterine devices, providing a diversity of delivery methods with varying durations of efficacy. These methods are effective and safe with minimal serious side effects or adverse events in healthy young women. However, hormonal methods are frequently discontinued due to changes in personal relationships, costs and side effects such as unscheduled vaginal bleeding or spotting, migraine headaches, nausea and weight gain. Of women who take daily oral hormonal contraceptives, 38% are reported to discontinue them within 12 months, principally due to unacceptable side effects. Many couples do not need or want to use or cannot afford daily hormonal or long acting methods such as implants or intrauterine devices. Fifty percent of women below the age of 40 report use of three to four different contraceptive methods during their reproductive life.

After discontinuing a hormonal method or during periods of infrequent and sporadic intercourse, women and men use on demand barrier contraceptives such as male and female condoms, spermicides, vaginal diaphragms with spermicide and withdrawal for unanticipated and unplanned intercourse. These methods have a high failure rate, are not discreet, can be uncomfortable, reduce the pleasure of the sex act, and are frequently neglected in the heat of the moment. The Pearl Index for pregnancy with unprotected intercourse is 80 per 100 women years' exposure. Typical user failure rates are 16.4 for male condoms and 13.4 for withdrawal [Polis Guttmacher Institute 2016]. Unprotected intercourse and contraceptive failures are associated with the 50% unintended pregnancy rate in 6.1 million pregnancies in 2011 in the United States [Guttmacher Institute 2016]. The abortion rate was 42% of the unintended pregnancies excluding spontaneous miscarriage. Non-hormonal methods are less effective than are the hormonal methods in preventing pregnancy.

Emergency contraception, a hormonal method used after unprotected intercourse, has a variable efficacy based on time from intercourse to use and is associated with an increased incidence of pregnancy with subsequent acts of intercourse. Also, many women believe that emergency contraception is after the fact and too late, and thus they do not use the method.

Levonorgestrel 1.5 mg is an emergency contraceptive (Plan B, Teva Pharmaceuticals, Parsippany, NJ). Taken within 72 hours of unprotected intercourse it has a variable efficacy ranging from a 38 to 95% reduction in expected pregnancies. The further from intercourse when taken, the higher the pregnancy incidence, and the product is ineffective after 72 hours following intercourse or if taken after the onset of the pituitary luteinizing hormone surge. However, the time of intercourse related to the woman's ovarian endocrine cycle (luteinizing hormone surge) and window of fertility is unknown or estimated in many instances. Women using emergency contraception are counseled not to have subsequent intercourse in that menstrual cycle since ovulation and pregnancy may occur after a temporary delay in ovulation due to the treatment.

A single dose of levonorgestrel taken prior to the LH surge results in both a delay for two days in the appearance of the LH surge and a reduction in the peak serum LH level with a subsequent five day delay in ovulation.

Levonorgestrel alone taken when the ovarian follicle reached a mean diameter of 18 mm had limited to no effect on delaying follicle rupture compared to placebo. This result could have been due to the administration of levonorgestrel after the onset of the pituitary LH surge that results in follicle rupture. Levonorgestrel inhibits, blunts or obtunds the LH surge when taken before the surge begins, but has no effect on LH dynamics after the onset of the surge.

The LH surge also up regulates intrafollicular cyclooxygenase-2 enzyme that is involved in the synthesis of prostaglandin E-2 a critical pathway involved in follicle rupture and ovulation in primates. Meloxicam is a known cyclooxygenase inhibitor that delays and inhibits follicle rupture in primates. Meloxicam alone given to monkeys for five days in a model of emergency contraception was allegedly effective but when taken daily with frequent intercourse was ineffective in preventing pregnancy. Meloxicam alone administration to normal women for five days with an 18 mm follicle delayed follicle rupture based on ultrasound evaluation by five days. Meloxicam has no effect on the LH surge, but it inhibits intrafollicular PTGS2, thus reducing PGE2. In monkeys, meloxicam has been allegedly shown to inhibit ovulation and reduced pregnancy rates in models of emergency contraception. Meloxicam, taken daily for five days, has allegedly been shown to inhibit ovulation in 27% of normal women but also allegedly had a 23% occurrence of an unruptured follicle for 6 days following treatment.

All female contraceptives have unique advantages and disadvantages. The attributes of the various methods allow women to switch from one method to another depending upon their personal preferences and current lifestyle. The average U.S. woman uses four different contraceptive methods during her reproductive life. Changing contraceptive methods frequently can leave gaps in the use of highly effective daily hormonal methods. Also, infrequent intercourse due to personal life style or changes in relationships is common. Thirty percent of sexually active women in the U.S. have intercourse less than three times a year, with another 30% reporting intercourse less than three times per month. Such women are less likely to use a daily hormonal contraceptive, but, in the absence of hormonal contraception, they are at high risk of an unwanted pregnancy.

There is thus a need in the art for a discreet, oral on demand contraceptive medication that can be taken immediately before or after intercourse, can be used for up to six act of intercourse during one menstrual cycle, is highly effective and has limited side effects. Such a medication and a method for its use would represent a significant advance for women with infrequent intercourse and those unable or unwilling to use a continuous hormonal method.

### SUMMARY OF THE INVENTION

It is to be understood that both the present general description of the embodiments and the following detailed description are exemplary, and thus do not restrict the scope of the embodiments.

In one aspect, the present invention meets the above-described need with a medication and method for its use that blocks or alters two major biologic pathways in order to inhibit or delay ovulation.

In another aspect, the invention provides a method of contraception in a female subject at risk of initiating an unwanted pregnancy, comprising administering to the female subject an effective amount of a combination of a progesterone receptor agonist and a cyclooxygenase-2 inhibitor in each of two doses, wherein the first dose is administered within about 24 hours before or after a first act of sexual intercourse, and wherein the second dose is administered between about 36 hours and about 60 hours after the first dose. In some embodiments, the second dose is administered about 48 hours after the first dose. In some embodiments, the second dose can be taken proximate in time to a second act of sexual intercourse.

In some embodiments, the first dose is taken within about 24 hours before the first act of sexual intercourse.

In some embodiments, the first dose is taken within about 24 hours after the first act of sexual intercourse.

In some embodiments, the inventive method further comprises administering at least one additional dose of said combination after administering the second dose.

In some embodiments, the progestin is levonorgestrel, and the cyclooxygenase-2 inhibitor is meloxicam.

In some embodiments, the female subject has three or fewer acts of intercourse within any one week of her menstrual cycle and six or fewer acts of intercourse within her menstrual cycle.

In some embodiments, each dose comprises from about 0.75 mg to about 3.0 mg of levonorgestrel and from about 7.5 mg to about 30 mg of meloxicam.

In some embodiments, each dose is administered orally.

In some embodiments, each administered dose is in the form of a slow-release oral tablet.

In some embodiments, the tablet for the first dose is identical to the tablet for the second dose.

In some embodiments, the slow-release oral tablet is formulated to include one or more polymers or waxes.

In some embodiments, the polymer(s) include biodegradable polyorthoesters or polyanhydrides.

In some embodiments, the doses are administered buccally, parenterally, transdermally, vaginally, or via a uterine route.

In some embodiments, the progestin is levonorgestrel and the cyclooxygenase-2 inhibitor is selected from the group consisting of indomethacin, diclofenac, naproxen, ibuprofen, nimesulide, ketoprofen and piroxicam.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The skilled artisan will understand that the drawings, described below, are for illustration purposes only. The drawings are not intended to limit the scope of the present teachings in any way.

FIG. 1 shows how levonorgestrel and meloxicam can act sequentially in the days before ovulation and the corresponding relationships between the LH surge and the appearances of progesterone and PGE2. The LH surge (purple) increases intrafollicular levels of progesterone (red) and PGE2 (blue). These changes occur during the 40 hours before ovulation. Levonorgestrel blocks or attenuates the luteinizing hormone (LH) surge. Meloxicam blocks PGE2 synthesis within the follicle.

FIG. 2 shows an overview of a clinical study design described in Example 1.

FIG. 3A shows a timeline of ovulation in relation to the days of the menstrual cycle as it occurs normally in the absence of contraceptive treatment. Blood concentration profiles for LH (purple) and PGE2 (blue) and concentration profiles for metabolites estrone conjugates (green) and pregnanediol glucuronide (red), assayed in urine samples as indicators of ovulation, are shown.

FIG. 3B shows the delay in ovulation, as associated with delays in spiking of LH and PGE2 blood concentrations, with administration of two doses of a combination of levonorgestrel and meloxicam, the two doses being about 48 hours apart.

### DETAILED DESCRIPTION OF THE INVENTION

There has not been a new or innovative approach to female contraception since “The Pill” in 1960. That medication was/is required to be taken daily, irrespective of the frequency of intercourse the woman experiences/wants. Being a hormonal-based medication, “The Pill” thus exposes a significant proportion of women (who have infrequent intercourse) to an unnecessary level of hormonal intake versus the intended outcome: reduced risk of pregnancy. The compositions and methods herein addresses a major unmet need for an effective, discreet and safe on demand female-controlled oral contraceptive method for women who do not wish to be exposed to continuous hormones. In addition, compositions and methods herein do not interfere with the sex act as do barrier contraceptives. The combination of these parameters are wholly consistent with providing women who do not have frequent intercourse a contraceptive giving them control over the timing of taking the medication in advance of the act of intercourse; the frequency with which they need to take the contraceptive; reducing their exposure to hormonal medication; and still having a contraceptive giving a strong reduction in the occurrence of pregnancy.

The compositions and methods herein provide an intervention specifically aimed at inhibiting two major pathways involved in ovulation to effectively prevent pregnancy when used on demand. The compositions and methods herein combine inhibition of the pituitary LH surge and intrafollicular PGE2 synthesis, each of which are critical to the process of ovulation, to provide a contraceptive method to be used with infrequent or unexpected acts of intercourse, and thereby provide a reduction of risk of pregnancy closer to that of daily hormonal contraceptive use but without the requirement to take unnecessarily a daily hormonal medication.

In some embodiments, the compositions and methods herein can provide a new approach for female contraception improving on current methods in the following ways:
reduces menstrual/abdominal cramping, reduces unscheduled endometrial bleeding and reduces menstrual blood loss in both normal and heavy bleeding women
will delay ovulation by 7 days by affecting two separate pathways involved in ovulation
administering the combination dose twice within 48 hours increases the delay in ovulation during the window of fertility
allows for planned and unplanned intercourse with a 24-hour efficacy around the sex act.
repetitive dosing used for infrequent acts of intercourse reduces common side effects of continuous hormonal contraception
more effective than typical user failure rates of combination oral contraceptives
efficacy significantly better than non-hormonal coitally related methods condom or withdrawal
linkage to the sexual act enhances compliance with the method
no interference with the sex act due to the large 24 hours window for efficacy
no impact on the couple's pleasure before and during the sex act
being oral provides a female-controlled discreet method used with unanticipated intercourse; and
minimal disruption in the woman's menstrual cycle related to frequency of use.

Reference will now be made in detail to embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, the term “about” means at most plus or minus 10% of the numerical value of the number with which it is being used.

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1st ed., 1992); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4th ed., 2000).

One skilled in the art may refer to general reference texts for detailed descriptions of known techniques discussed herein or equivalent techniques. These texts can include Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

It is contemplated that any method or composition described herein can be implemented with respect to any other method or composition described herein.

In one embodiment, the invention provides a method of contraception in a female subject at risk of initiating an unwanted pregnancy, comprising administering to the female subject an effective amount of a combination of a progesterone receptor agonist and a cyclooxygenase-2 inhibitor in each of two doses, wherein the first dose is administered within about 24 hours before or after a first act of sexual intercourse, and wherein the second dose is administered at least about 48 hours after the first dose.

In some embodiments, the invention provides a combination of a) levonorgestrel, a synthetic orally active progestogen and b) meloxicam, a non-steroidal anti-inflammatory drug (NSAIDs). Without being bound by theory, both drugs can inhibit ovulation via a) inhibition of the pituitary luteinizing hormone (LH) release and b) inhibition of the LH induced progesterone response in the ovarian follicle with induction of the cyclooxygenase 2 (COX-2) enzyme that synthesizes prostaglandin E2 resulting in creation of the tunnel for oocyte (egg) escape from the follicle, respectively. In some aspects, endometrial PGE2 is increased just before menstruation and causes dysmenorrhea or intermittent cramping that occurs before and during menstruation.

In some embodiments, the invention possess one or more non-limiting additional advantages. In some embodiments, addition of meloxicam to levonorgestrel increases the duration of contraceptive efficacy by inhibiting the post LH induction of follicular cyclooxygenase 2 (COX-2). In some embodiments, meloxicam inhibits menstrual or abdominal cramps, unscheduled endometrial bleeding, and reduces normal and heavy menstrual bleeding. In some embodiments, meloxicam inhibits the development of the follicular tunnel which allows escape of the egg at ovulation. In some embodiments, the invention reduces dysmenorrhea-premenstrual and menstrual cramps. In some embodiments, the invention leads to reduction in blood loss with menstruation particularly in women with heavy menstrual bleeding but normal menstrual blood loss will show a reduction.

The term “female subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

In accordance with the invention, a “therapeutically effective amount” or “effective amount” of a combination of a progesterone receptor agonist and a cyclooxygenase-2 inhibitor is administered to the subject. As used herein a “therapeutically effective amount” or “effective amount” is an amount sufficient to reduce the incidence of pregnancy when the combination is used as a contraceptive according to an embodiment of the invention.

The “Pearl Index” is a commonly used technique for reporting the effectiveness of a birth control method. The Pearl Index is calculated as follows:

$$Pearl Index = \frac{( N umber of pregnancies ) ( 12 )}{( N umber of women ) ( n umber of months )} \times 100$$

The “number of months” refers to the number of months of exposure by women in the particular study. Alternatively, given that a fertile woman on average has 13 menstrual cycles per year, “number of months” can be replaced by “number of menstrual cycles” and “12” can be replaced by “13.” For a contraceptive method, a lower Pearl Index corresponds to a lower incidence of unwanted pregnancy.

In some embodiments, the administration of the doses of the progesterone receptor agonist and cyclooxygenase-2 inhibitor effects a delay in ovulation. Without being bound by theory, it is believed that the delay in ovulation is due to inhibition of the pituitary LH surge and intrafollicular PGE2 synthesis.

In some embodiments, the administration of the first and second doses delays ovulation by at least about 7 days, measured in time from the administration of the first dose. In some embodiments, the administration of the first and second doses delays ovulation by at least about 8, 9, 10, 11, or 12 days, measured in time from the administration of the first dose.

In some embodiments, the method reduces the need for continuous hormonal contraceptives and can be used instead of male or female condoms, diaphragms or withdrawal approaches.

In some embodiments, the method is used in combination with other contraceptive methods, such as male or female condoms, diaphragms or withdrawal approaches. In some embodiments, the female subject can be in the process of transiting off of continuous hormonal contraceptives or other contraceptive methods. In some embodiments, the female subject has infrequent sexual intercourse. In some embodiments, the sexual intercourse does not include use of a barrier contraceptive or withdrawal prior to ejaculation.

### Progesterone Receptor Agonist

The progesterone receptor agonist for use in the methods of the invention is not particularly limiting. In some embodiments, the progesterone receptor agonist is selected from the group consisting of levonorgestrel (e.g., about 0.75 mg to about 4.5 mg), ulipristal acetate (e.g., about 5.0 to 10 mg), mifepristone (e.g., about 2.5 mg), norethindrone (e.g., about 0.350 mg to about 0.5 mg), gestodene, norethindrone acetate (e.g., about 1.0 mg), desogestrel (e.g., about 0.150 mg), etonogestrel (e.g., about 0.250 mg), promegestone (e.g., about 0.5 mg), dienogest (e.g., about 1.0 mg), nomegestrol acetate (e.g., about 1.25 to about 5.0 mg) and drospirenone (e.g., about 3.0 mg).

In one embodiment, the progesterone receptor agonist is levonorgestrel. Levonorgestrel is believed to inhibit or obtund the pituitary luteinizing hormone surge when taken before the surge begins, thus delaying or inhibiting ovulation. The progesterone receptor agonist dose can be administered in one or more compositions. In some embodiments, the progesterone receptor agonists is the same compound administered in the first and second dose. In some embodiments, the progesterone receptor agonist in the first dose is different from the progesterone receptor agonist in the second dose. In some embodiments, a combination of progesterone receptor agonists are administered in the first and/or second doses.

In some embodiments, the progesterone receptor agonist is levonorgestrel and each dose comprises between about 0.75 mg and about 3.0 mg of levonorgestrel.

### Cyclooxygenase-2 Inhibitor

In some embodiments, the cyclooxygenase-2 inhibitor is selected from indomethacin, diclofenac, naproxen, ibuprofen, nimesulide, ketoprofen, piroxicam, meloxicam, and combinations thereof.

The amount of the inhibitor present in the pharmaceutical preparation can vary depending on the inhibitor chosen. In some embodiments, the following dose ranges of the cyclooxygenase-2 inhibitor are suitable: Indomethacin: 50-400 mg; Diclofenac: 50-400 mg; Naproxen: 500-3000 mg; Ibuprofen: 500-5000 mg; Nimesulide: 100-500 mg; Ketoprofen: 100-500 mg; Piroxicam: 10-80 mg Meloxicam: 7-30 mg. The dose of the cyclooxygenase-2 inhibitor can be administered in one or more compositions.

Meloxicam is believed to not affect the luteinizing hormone surge but inhibit ovarian follicle prostaglandin E2 synthesis increased by the LH surge, preventing follicle rupture/ovulation.

Without being bound by theory, the combination of the progesterone receptor agonist and cyclooxygenase-2 inhibitor is thought to alter these two major ovulatory pathways by independent means, resulting in an enhanced delay or inhibition of ovulation, increasing contraceptive efficacy.

These two pathways are sequentially activated with the onset of the luteinizing hormone surge and ovulation 48 hours later. Altering these two pathways can delay ovulation beyond the five days of sperm fertilizing capacity in the female reproductive tract. The compositions and methods herein can be used with a single or repetitive acts of intercourse within a single menstrual cycle.

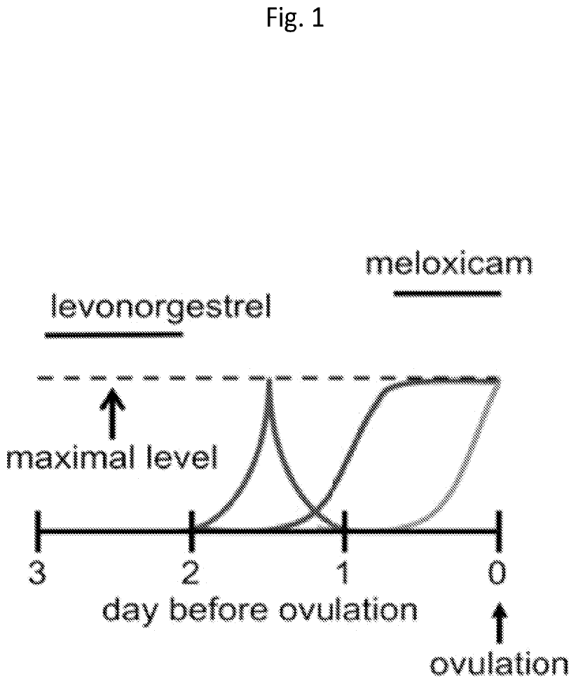

In one aspect, the invention provides a method for administering an effective amount of a combination of levonorgestrel and meloxicam. FIG. 1 shows how levonorgestrel and meloxicam can act sequentially in the days before ovulation and the corresponding relationships between the LH surge and the appearances of progesterone and PGE2. In one embodiment of the present invention, two oral doses of a combination of levonorgestrel and meloxicam are administered about 48 hours apart and during a woman's fertile period to achieve a delay in ovulation of greater than about seven days. This result cannot be explained by a simple addition of the effects of levonorgestrel and meloxicam and provides for an effective contraceptive.

Highly effective continuous hormonal contraceptive is generally recommended for sexually active women having frequent intercourse. In some embodiments, the compositions and methods herein can have equal contraceptive efficacy with fewer side effects in women who have sexual intercourse less frequently.

In one embodiment, the method comprises administering to the female subject a first dose comprising an oral dosage form comprising an effective amount of a combination of levonorgestrel and meloxicam within about 24 hours before or after sexual intercourse, followed by administering a second dose comprising an oral dosage form comprising an effective amount of a combination of levonorgestrel and meloxicam between about 36 hours and about 60 hours after the first dose.

In some embodiments, the female subject engages in a second or multiple acts act of sexual intercourse and the first and second doses are effective at preventing pregnancy.

In accordance with the invention, the second dose is taken regardless of whether or not the female subject engages in a second, third, or fourth, etc. act of sexual intercourse. In some embodiments, the female subject engages in one or more additional acts of sexual intercourse within about 48 hours from the second dose. In such embodiments, it is not necessary for the female subject to administer an additional dose of the active agents in addition to the first and second dose.

In some embodiments, the female subject engages in an act of sexual intercourse after about 48 hours from the second dose. For acts of sexual intercourse occurring about 48 hours after the second dose, the method further comprises administering a third dose within 24 hours of the additional act of sexual intercourse and a fourth dose between about 36 hours and about 60 hours after the third dose. Although there is no maximum number of doses in a menstrual cycle that can be administered to the female subject, it is preferable to not administer more than 8 doses in a single menstrual cycle in order to avoid any significant side effects. In some embodiments, menstrual cycle delay could occur with unscheduled vaginal bleeding with the use of >6 doses per month.

In some embodiments, the female subject engages in three or fewer acts of sexual intercourse within one week.

In some embodiments, the female subject has sexual intercourse six times or less per menstrual cycle. In some embodiments, the female subject has sexual intercourse five times or less, four times or less, three times or less, two times or less, or one time or less per menstrual cycle.

In certain embodiments of the invention, each dose is comprises between about 0.75 mg and about 3.0 mg of levonorgestrel and between about 7.5 mg and about 30 mg of meloxicam. In some embodiments, the active agents are formulated and administered as a slow-release oral tablet or capsule. In some embodiments, each dose can include about 1.5 mg levonorgestrel and about 15 mg meloxicam.

Sexual intercourse in humans can occur at any time in the woman's menstrual cycle in contrast to animals where intercourse is limited to the days immediately preceding ovulation (estrous). Women are most fertile on the four days preceding ovulation and the day of ovulation, the window of fertility, where unprotected intercourse results in >90% of all pregnancies. Intercourse on other days of the woman's menstrual cycle can also result in pregnancy, and, therefore, the product must be effective and safe with repetitive use.

In some embodiments, the first dose is administered within about 12 hours before or about 12 hours after intercourse.

In some embodiments, the method can be used up to six times within a menstrual cycle. Repetitive acts of intercourse over several days can be managed by administering doses of the active agents about every 48 hours.

In some embodiments, the female subject is fertile and has infrequent intercourse or who does not wish to use continuous contraceptive hormones. In some embodiments, the female subject has sexual intercourse six times or less per menstrual cycle. In some embodiments, the female subject has more than six acts of sexual intercourse per menstrual cycle.

Levonorgestrel and meloxicam are almost completely absorbed from the gastrointestinal tract. Both have highly variable serum concentration maximums, and have estimated serum half-lives of 27.5 and 20 hours, respectively. Levonorgestrel is metabolized principally by the liver enzyme CYP3A4. Meloxicam is metabolized principally via CYP2C9 but CYP3A4 is also involved in its metabolism. CYP3A4 inhibitors or competitors are known to reduce levonorgestrel blood levels and its contraceptive efficacy. There are no studies of drug-drug interaction between levonorgestrel and meloxicam.

In some embodiments, the compositions and methods herein have a typical use contraceptive efficacy comparable to existing hormonal methods, with five or fewer pregnancies per 100 women-years of use (Table 1).

TABLE 1
Summary of exemplary contraceptives¹⁷

| | | | Pregnancies/ |
|---|---|---|---|
| Type of | | Major | 100 woman/ |
| Contraceptive | Examples | disadvantages | year |
| Long acting; | IUD; Hormonal | Some methods | <1 |
| reversible or | Implant | irreversible; side | |
| irreversible | Surgical | effects of | |
| | Sterilization | long-term | |
| | | hormone therapy | |
| Continuous dosing | Hormonal: Pill, | Some require | <1 to 9 |
| | Patch, | regular use (pill); | |
| | Injectable, | side | |
| | Vaginal Ring | effects of long- | |
| | | term hormone | |
| | | therapy | |
| Use during sexual | Male/Female | Must be used | 12-28 |
| intercourse | Barriers, | properly at each | |
| | Diaphragm, | act of | |
| | Withdrawal, | intercourse | |
| | Spermicide | | |
| Emergency | Hormonal: | Increased risk of | NA; emergency |
| contraceptive | Plan B, Ella | pregnancy with | use only |
| | | subsequent act of | |
| | | intercourse or | |
| | | delay | |
| | | between | |
| | | intercourse and | |
| | | drug | |
| | | administration | |

### Treatment Regimen

The treatment regimen of administering an effective amount of the progesterone receptor agonist and an effective amount of the cyclooxygenase-2 inhibitor is not limiting.

The combination of progesterone receptor agonist and cyclooxygenase-2 inhibitor can be administered alone or in combination with one or more additional active pharmaceutical agents.

As provided herein, the female subject is administered at least two doses of a progesterone receptor agonist and cyclooxygenase-2 inhibitor. The dose of progesterone receptor agonist and cyclooxygenase-2 inhibitor can be administered in a single composition or in multiple compositions. Thus, the combined administration includes co-administration, using separate formulations or a single pharmaceutical formulation, and consecutive administration in either order, wherein preferably there is a time period while both active agents simultaneously exert their biological activities. In some embodiments, the dose of progesterone receptor agonist and cyclooxygenase-2 inhibitor is administered in a single composition. In some embodiments, the single composition comprises and orally administered slow or delayed release tablet or capsule.

The effective amount of the progesterone receptor agonist can be administered in one or more compositions, and the effective amount of the cyclooxygenase-2 inhibitor can also be administered in one or more compositions.

The route of administration of the progesterone receptor agonist and cyclooxygenase-2 inhibitor can be the same or different. In some embodiments, the progesterone receptor agonist or cyclooxygenase-2 inhibitor can be administered via multiple routes.

In some embodiments, the progesterone receptor agonist is administered orally, buccally, parenterally, transdermally, vaginally, or via a uterine route.

In some embodiments, the cyclooxygenase-2 inhibitor is administered orally, buccally, parenterally, transdermally, vaginally, or via a uterine route.

In some embodiments, the progesterone receptor agonist and cyclooxygenase-2 inhibitor can be administered together in the same composition or in separate compositions. In some embodiments, the progesterone receptor agonist and cyclooxygenase-2 inhibitor are administered in separate compositions.

The formulations for administration are not limiting and can include, e.g., immediate release, extended release (i.e., slow release over an extended period of time), controlled release, and burst release formulations. In some embodiments, the active agents are released over a period of hours, such as over 2, 4, 6, 8, 10 or 12 hours. In some embodiments, the active agents are released over 12 hours with an initial burst.

In some embodiments, the amount of progesterone receptor agonist and/or the cyclooxygenase-2 inhibitor administered to the subjects can be determined according to the subject's body weight.

For example, in some embodiments, the dosage of levonorgestrel can be between about 0.015 and 0.06 mg/kg body weight of the subject to be treated. In some embodiments, the dosage of meloxicam can be between about 0.1 and 0.6 mg/kg body weight of the subject to be treated. In some embodiments, the effective dosage of levonorgestrel is between about 0.015-0.025, 0.016-0.022 or 0.018-0.02 mg/kg body weight. In some embodiments, the effective dosage of meloxicam is between about 0.1-0.25, 0.14-0.22 or 0.15-0.2 mg/kg body weight. In some embodiments, the effective amount of meloxicam is in the range of about 7.5 mg to about 30 mg per dose. For example, some methods entail administration to a subject in need of treatment with a meloxicam dosage of about 7.5 mg, 9 mg, 11 mg, 13 mg, 15 mg, 17 mg, 19 mg, 21 mg, 23 mg, 25 mg, 27 mg, or 30 mg per dose. In some embodiments, subjects are administered a dosage of between about 14 mg to about 20 mg of meloxicam per dose. In some embodiments, subjects are administered a dosage of about 14 to about 17 mg meloxicam. In some embodiments, the meloxicam dosage administered is about 15 mg. The dosage of the levonorgestrel and the dosage of the meloxicam to be administered is not necessarily limiting.

In some embodiments, the levonorgestrel is administered at a dose of from about 0.5 mg to about 2.5 milligrams (mg), from about 0.7 mg to about 2 mg, from about 0.8 mg to about 1.8 mg, from about 0.9 mg to about 1.7 mg, from about 1 mg to about 1.7 mg, from about 1.2 mg to about 1.7 mg, from about 1.3 mg to about 1.6 mg, from about 1.4 mg to about 1.6 mg, or about 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg or more per dose.

In some embodiments, the subject female to be treated in accordance with the inventive methods have achieved contraception with a Pearl Index of about seven or less to about 12, e.g., 12 or less, 11 or less, 10 or less, 9 or less, 8 or less, or 7 or less. In some embodiments, the subject female has achieved these results for multiple years of use of the inventive methods.

In some embodiments, the levonorgestrel/meloxicam combination slow-release tablets or capsules can be provided in a kit comprising a multiple dose dispensing unit such as a blister pack or dial pack type dispenser, preferably with instructions (e.g., take a first dose within about 24 hours before or within 24 hours after sexual intercourse and take a second dose between about 36 hours and 60 hours after the first dose). In some embodiments, the instructions are printed on the dispenser.

One skilled in the art can also readily determine an appropriate dosage regimen for administering the combination of active ingredients of the invention to a given subject. For example, the subject compound(s) or composition(s) can be administered once from 24 hours before intercourse to 24 hours after intercourse during the woman's fertile period, then with subsequent instances of intercourse during the same menstrual cycle, with this regime to be repeated over multiple menstrual cycles. In some dosage regimens, the compound(s) or composition(s) are administered orally. Where a dosage regimen comprises multiple administrations, it is understood that the effective amount of the compound(s) or composition(s) administered to the subject can comprise the total amount of the compound(s) or composition(s) administered over the entire dosage regimen. The exact amount can depend on the subject woman's health history, her weight, previous responses to the subject compound(s) or composition(s), and will be ascertainable by a person skilled in the art using known methods and techniques for determining effective doses.

### Compositions

The active agents, e.g., levonorgestrel and meloxicam, of the present invention can be administered alone or in combination with one or more active pharmaceutical agents.

Solid dosage forms for oral administration may include capsules, tablets, pills, powders, gelcaps and granules. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such as magnesium stearate and microcrystalline cellulose. In the case of capsules, tablets and pills, the dosage forms may also comprise buffering agents. Tablets and pills can additionally be prepared with enteric coatings and other release-controlling coatings.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferably, in a certain part of the intestinal tract, optionally in a delayed manner. Examples of embedding compositions which can be used include polymeric substances and waxes.

Liquid dosage forms for oral administration may include pharmaceutically acceptable emulsions, microemulsions, solutions, suspensions, syrups and elixirs containing inert diluents commonly used in the art, such as water, isotonic solutions, or saline. Such compositions may also comprise adjuvants, such as wetting agents; emulsifying and suspending agents; sweetening, flavoring and perfuming agents.

Suppositories for rectal administration of the compound(s) and composition(s) of the invention can be prepared by mixing the drugs with a suitable non-irritating excipient, such as cocoa butter and polyethylene glycol which are solid at ordinary temperature but liquid at the rectal temperature and will, therefore, melt in the rectum and release the drug.

In some embodiments, the progesterone receptor agonist and/or the cyclooxygenase-2 inhibitor is administered by an intravaginal ring. In some embodiments, the intravaginal ring is formulated for extended release or immediate release. In some embodiments, therapeutic levels of the progesterone receptor agonist and/or the cyclooxygenase-2 inhibitor are released over a period of days by a single ring, e.g., 2 days, 3 days, 5 days, 6 days, 7 days, 10 days, 2 weeks, or longer. In some embodiments, the treatment regimen incorporates a series of rings wherein a first ring is administered that supplies a first dose of progesterone receptor agonist and/or cyclooxygenase-2 inhibitor followed by the administration of one or more additional intravaginal rings in sequence that provide, e.g., a second dose, third dose, etc. of the progesterone receptor agonist and/or cyclooxygenase-2 inhibitor.

Dosage forms for topical or transdermal administration of a compound can further include ointments, pastes, creams, lotions, gels, powders, solutions, sprays, inhalants or patches. Transdermal patches have the added advantage of providing controlled delivery of active compound to the body. Such dosage forms can be made by dissolving or dispersing the compound in the proper medium. Absorption enhancers can also be used to increase the flux of the compound across the skin. The rate can be controlled by either providing a rate controlling membrane or by dispersing the compound in a polymer matrix or gel. The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In one embodiment, the progesterone receptor agonist and/or the cyclooxygenase-2 inhibitor is delivered transdermally. The term “transdermal delivery” as used herein means administration of the pharmaceutical composition topically to the skin, wherein the active ingredient or its pharmaceutically acceptable salts will be percutaneously delivered in a therapeutically effective amount.

In some embodiments, the composition to be applied transdermally further comprises an absorption enhancer. The term “absorption enhancer” as used herein means a compound which enhances the percutaneous absorption of drugs. These substances are sometimes also referred to as skin-penetration enhancers, accelerants, adjuvants and sorption promoters. Various absorption enhancers are known to be useful in transdermal drug delivery. U.S. Pat. Nos. 5,230,897, 4,863,970, 4,722,941, and 4,931,283 disclose some representative absorption enhancers used in transdermal compositions and for topical administration. In some embodiments, the absorption enhancer is N-lauroyl sarcosine, sodium octyl sulfate, methyl laurate, isopropyl myristate, oleic acid, glyceryl oleate or sodium lauryl sulfoacetate, or a combination thereof. In some embodiments, the composition contains on a weight/volume (w/v) basis the absorption enhancer in an amount of about 1-20%, 1-15%, 1-10% or 1-5%. In some embodiments, to enhance further the ability of the therapeutic agent(s) to penetrate the skin or mucosa, the composition can also contain a surfactant, an azone-like compound, an alcohol, a fatty acid or ester, or an aliphatic thiol.

In some embodiments, the transdermal composition can further comprise one or more additional excipients. Suitable excipients include without limitation solubilizers (e.g., C₂-C₈ alcohols), moisturizers or humectants (e.g., glycerol [glycerin], propylene glycol, amino acids and derivatives thereof, polyamino acids and derivatives thereof, and pyrrolidone carboxylic acids and salts and derivatives thereof), surfactants (e.g., sodium laureth sulfate and sorbitan monolaurate), emulsifiers (e.g., cetyl alcohol and stearyl alcohol), thickeners (e.g., methyl cellulose, ethyl cellulose, hydroxymethyl cellulose, hydroxypropyl cellulose, polyvinylpyrrolidone, polyvinyl alcohol and acrylic polymers), and formulation bases or carriers (e.g., polyethylene glycol as an ointment base). As a non-limiting example, the base or carrier of the composition can contain ethanol, propylene glycol and polyethylene glycol (e.g., PEG 300), and optionally an aqueous liquid (e.g., isotonic phosphate-buffered saline).

Exemplary pharmaceutically acceptable carriers include carriers suitable for oral, intravenous, subcutaneous, intramuscular, intracutaneous, and the like administration. Administration in the form of creams, lotions, tablets, dispersible powders, granules, syrups, elixirs, sterile aqueous or non-aqueous solutions, suspensions or emulsions, and the like, is contemplated.

For the preparation of oral liquids, suitable carriers include emulsions, solutions, suspensions, syrups, and the like, optionally containing additives such as wetting agents, emulsifying and suspending agents, sweetening, flavoring and perfuming agents, and the like.

For the preparation of fluids for parenteral administration, suitable carriers include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized, for example, by filtration through a bacteria-retaining filter, by incorporating sterilizing agents into the compositions, by irradiating the compositions, or by heating the compositions. They can also be manufactured in the form of sterile water, or some other sterile injectable medium immediately before use. The active compound is admixed under sterile conditions with a pharmaceutically acceptable carrier and any needed preservatives or buffers as may be required.

The treatments may include various “unit doses.” Unit dose is defined as containing a predetermined quantity of the therapeutic composition calculated to produce the desired responses in association with its administration, e.g., the appropriate route and treatment regimen. Also of importance is the subject to be treated, in particular, the state of the subject and the protection desired.

In some embodiments, pharmaceutical compositions of the present invention comprise an effective amount of a progesterone receptor agonist and/or a cyclooxygenase-2 inhibitor dissolved or dispersed in a pharmaceutically acceptable carrier. The phrases “pharmaceutical” or “pharmacologically acceptable” refer to molecular entities and compositions that do not produce an adverse, allergic or other untoward reaction when administered to an animal, such as, for example, a human, as appropriate. The preparation of a pharmaceutical composition that contains at least one progesterone receptor agonist and/or cyclooxygenase-2 inhibitor will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by the FDA.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The progesterone receptor agonist and/or cyclooxygenase-2 inhibitor may comprise different types of carriers depending on whether it is to be administered in solid, liquid or aerosol form. The compound(s) and/or composition(s) of the present invention can be administered intradermally, transdermally, intravaginally, intrarectally, topically, intramuscularly, subcutaneously, mucosally, orally, topically, locally, infusion, continuous infusion, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., decomposition in the stomach. Examples of stabilizers for use in the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle composition that includes progesterone receptor agonist and/or a cyclooxygenase-2 inhibitor, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the progesterone receptor agonist and/or a cyclooxygenase-2 inhibitor may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

In certain embodiments, pharmaceutical compositions may comprise, for example, at least about 0.1% of an active compound or compounds. In other embodiments, active compound(s) may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound(s). Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments of the present invention, the progesterone receptor agonist and/or a cyclooxygenase-2 inhibitor are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier or they may be enclosed in hard- or soft-shell gelatin capsule, or they may be compressed into tablets, or they may be incorporated directly with the food of the diet.

In some embodiments, the progesterone receptor agonist and/or cyclooxygenase-2 inhibitor are formulated to be contained in a food product, and the drugs are delivered by consuming the food product. In some embodiments, the food product comprises gummy candy products. In some embodiments, the food products are cookies. The food product that can be used is not limiting.

In certain embodiments, the active compounds may be incorporated with excipients and used in the form of ingestible tablets, buccal tables, troches, capsules, elixirs, suspensions, syrups, wafers, and the like. See, e.g., U.S. Pat. Nos. 5,641,515; 5,580,579 and 5,792,451, each of which is specifically incorporated herein by reference in its entirety. The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent, methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparations and formulations.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

In further embodiments, the progesterone receptor agonist and/or cyclooxygenase-2 inhibitor may be administered via a parenteral route. As used herein, the term “parenteral” includes routes that bypass the alimentary tract. Specifically, the pharmaceutical compositions disclosed herein may be administered for example, but not limited to intradermally, transdermally, intramuscularly, subcutaneously, or intraperitoneally. See, e.g., U.S. Pat. Nos. 6,7537,514; 6,613,308; 5,466,468; 5,543,158; 5,641,515; and 5,399,363, which are each specifically incorporated herein by reference in their entireties.

### Examples

Example 1: Comparison of Levonorgestrel Pharmacokinetics (PK) when Used Alone or in Combination with Meloxicam.

Levonorgestrel 1.5 mg pharmacokinetics are variable with mean peak blood levels of 18 ng/mL, as determined using immunoassay methodology. Mean peak serum levonorgestrel levels of 18.3 and 10.2 ng/mL have been reported from two different laboratories, both using liquid chromatography and tandem mass spectrometry; this discrepancy is probably due to methodological differences. Meloxicam pharmacokinetics evaluated with specific gas liquid chromatography methodology found high variability in serum levels between individuals, with peak levels occurring after five or six hours depending upon food intake. Meloxicam 15 mg daily reaches a steady state concentration after three to five days.

Both levonorgestrel and meloxicam are metabolized by CYP3A4, a liver enzyme. Medications that inhibit CYP3A4 reduce levonorgestrel serum levels and contraceptive efficacy. The example herein describe a clinical study that is designed to compare levonorgestrel pharmacokinetics (PK) when used alone or in combination with meloxicam. The levonorgestrel PK outcomes are serum concentration maximum (Cmax) and the area under the curve (AUC) concentration from zero to 96 hours, and from zero to infinity. A significant difference in the levonorgestrel PK results with or without meloxicam is not anticipated.

The levonorgestrel PK outcomes are serum concentration maximum (Cmax) and the area under the curve (AUC) concentration from zero to 96 hours, and from zero to infinity. A significant difference in the levonorgestrel PK results with or without meloxicam is not anticipated.

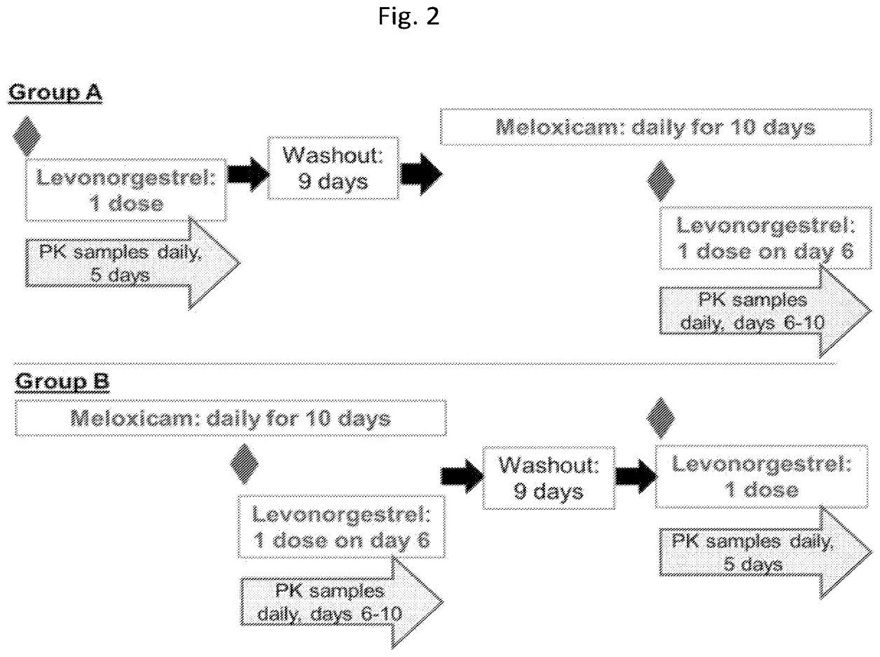

This will be an open label randomized two-drug crossover study to test for an interaction between two well-characterized FDA-approved drugs: levonorgestrel and meloxicam. The study will enroll 24 women between 20 and 35 years of age. To be enrolled, the potential participant must meet all inclusion and exclusion criteria. By signing an Institutional Review Board approved Informed Consent, she will indicate her willingness to be a participant in the study. The participant will then be randomized to either Treatment A or B, followed by a wash out period of 9-10 days and then crossed over to the second treatment. Treatment A is levonorgestrel 1.5 mg with a pharmacokinetic evaluation lasting five days followed by a wash out period of nine days and then meloxicam daily for ten days with levonorgestrel given on day six of treatment followed by the pharmacokinetic evaluation lasting five days. Treatment B will start with meloxicam 15 mg/day for ten days with levonorgestrel on day six of treatment with a pharmacokinetic evaluation of five days followed by a nine day wash out period and then levonorgestrel followed by a five day pharmacokinetic evaluation. The day of levonorgestrel administration in each treatment arm will be the start of a five day standard PK evaluation with peripheral vein blood draws scheduled before medication at time 0 and then at 0.5, 1, 2, 4, and 6±0.25 hours and then 24, 30, 48, 56, 72, 80, and 96±1.0 hours for a total of 13 blood draws. All blood samples will be allowed to clot at room temperature, centrifuged at 5000 rpm for 15 minutes and then the serum will be decanted and stored at −80° C. until shipped to the central laboratory for levonorgestrel analysis using a liquid chromatography tandem mass spectrometry method. All samples from the same participant will be analyzed in one assay to reduce inter-assay variability. The outcome will be levonorgestrel pharmacokinetic profiles compared between levonorgestrel alone and levonorgestrel plus meloxicam. The primary endpoints will be three pharmacokinetic parameters for levonorgestrel: Area under the curve (AUC) 0 to 96 hours, AUC 0 to infinity and concentration maximum (Cmax). The data will be log transformed and tested to determine if there is a statistically significant difference in each of these log transformed parameters, using a t-test based on mean ratios. The 90% confidence interval (CI) for the ratio of the geometric means will also be determined. A finding of equivalence will be defined if the ratio of the geometric means is contained in the interval of 0.70 to 1.43. The study design is shown in the schematic of FIG. 2.

### Example 2: Determination of Side Effects and Safety of Levonorgestrel Alone and Levonorgestrel Plus Meloxicam.

For the women in the clinical trial described in Example 1, reported side effects of levonorgestrel alone and in combination with meloxicam will be recorded. Null hypothesis: There will be no significant differences in reported side effects for each medication between the two treatments.

Side effects and/or symptoms will be captured using a daily menstrual diary card with the participant recording meloxicam and/or levonorgestrel taken and recording any symptoms or adverse events. Safety assessments will use significant changes in medical history, physical findings, vital signs, hematology, clinical chemistry (liver and renal function) and urine analysis between the enrollment and exit visits.

The anticipated result is that meloxicam will be associated with abdominal cramping or pain with nausea and headache in less than 10% of the participants during treatment. The relatively small sample size and limited exposure to meloxicam may not result in a significant incidence of vomiting, diarrhea or skin changes as side effects. Levonorgestrel use may result in unscheduled vaginal bleeding or spotting, with a change of ±2 days in the day of onset of the subsequent menstrual bleed in 20% of the participants. Nausea, abdominal pain and headache could occur with a range of 5 to 10% with use of levonorgestrel, and discomfort at the site of blood sampling could result in minor/moderate pain and soreness. Both of these findings are reduced by the addition of meloxicam. Adverse symptoms related to the study medication are anticipated to be mild, limited in duration, and temporally related to the study medications but should not result in discontinuation of the medication nor withdrawing from the study.

Each medication has its own adverse or side effect outcomes. Levonorgestrel is indicated as a contraceptive and meloxicam is used for relief of arthritis pain. Age and underlying disease in the older woman can contribute to meloxicam side effects and adverse outcomes. However, both medications have regulatory agency approval and have tolerable side effects in young healthy women. Serious adverse effects with levonorgestrel have not been reported. Serious adverse events associated with meloxicam are rare, usually involve the gastrointestinal tract, cardiovascular system, liver, or skin. Due to the limited exposure to each medication and the use of approved doses of each, no serious medication-related side effects are anticipated.

The common side effects listed for each medication are taken from the FDA approved package insert or a specific publication where the medication(s) was used in an On Demand fashion in women. For levonorgestrel 1.5 mg as a single dose, side effects were heavy menstrual bleeding 30.9%, nausea 13.7%, lower abdominal pain 13.3%, fatigue 13.3%, headache 10.3%, dizziness 9.6%, breast tenderness 8.2% and delay of menses >7 days in 4.5% in n=1359 women (Physician Label Plan B One Step® Barr Pharmaceuticals, Inc. Pomona, NY). Post marketing experience of levonorgestrel 1.5 mg found similar complaints with the additional complaints of vomiting and dysmenorrhea (Physician Label Plan B). Meloxicam 15 mg (Physician Label, Mobic®, Boehringer Ingelheim Pharmaceutics, Inc. Ridgefield, CT) adverse events reported from a 12 week rheumatoid arthritis placebo controlled trial reported incidence of abdominal pain 2.6%, dyspepsia 4.5%, nausea 3.8%, edema 4.5%, dizziness 3.8%, headache 8.3% and skin rash 0.6%. One clinical trial of oral meloxicam 15 mg for 5 days in normal women reported adverse events in 27.3% of the women with low abdominal pain in 13.6%, bloating in 4.5% and hot flushes in 9.0% (McCann et al., Contraception, (2013), 88:744-8). Meloxicam 15 mg taken daily for 18 days had an incidence of adverse events of 33.7% with upper respiratory infections being the most frequent adverse event, and there were no serious adverse events reported (Centers for Disease Control and Prevention, Atlanta, GA: Centers for Disease Control and Prevention, (2014)). A clinical trial of levonorgestrel plus placebo or meloxicam randomized 41 women to one or the other treatment and found that 22 women had no side effects at all 18. The occurrence of stomachache, low abdominal pain, headache, vaginal discharge, dizziness, sleepiness, diarrhea, and breast tenderness was not different between the remaining women in the two groups (Massai et al., Hum Reprod, (2007), 22:434-9).

Example 3: For Doses of a Combination of 1.5 mg Levonorgestrel with 15 mg Meloxicam, Determine the Interval Between the First Dose of Levonorgestrel Plus Meloxicam to Ovulation in Normal Women Compared to Placebo (Phase II).

There will be a delay of ≥7 days between the first dose of combination drug and the occurrence of ovulation compared to two days following placebo. The outcome will use the well characterized luteal-follicular shift that reflects formation of the corpus luteum with changes in urinary estrogen conjugates and pregnanediol glucuronide. These data are expected to establish that levonorgestrel plus meloxicam given in two doses is highly effective in delaying ovulation in normal women.

A single dose of levonorgestrel (LNG) taken prior to the LH surge results in both a delay and a reduction in the peak serum LH levels for two days with a subsequent 5-day delay in ovulation (Brache et al., Contraception, (2013), 88:611-8; Croxatto et al., Contraception, (2001), 63:111-21; Devoto et al., Fertility and sterility, (2005), 84:46-51). There is only one clinical study which directly compared LNG plus meloxicam (MEL) vs. LNG alone, the endpoint being the percentage of follicles with no rupture within five days of a single dose of either treatment (Massai et al., Hum Reprod, (2007), 22:434-9). This pilot study reported a single dose of LNG plus MEL given when the maximum follicle diameter was 17 mm delayed ovulation for 5 days in 70% of women but was not significantly different than the result obtained with LNG alone (50%) due to the small sample size of 10 participants. The same study gave LNG plus MEL to 31 women with a follicle diameter of ≥18 mm, and follicle rupture was delayed for 5 days in 39% of women receiving LNG plus Mel vs 16% of women receiving LNG alone (p=0.052). These data indicate that a single dose combination of LNG plus MEL is more likely to delay ovulation than LNG alone. This study is the only existing clinical study of LNG plus MEL in normal women, and the interpretation of the results therein is hampered by small sample sizes and use of a single dose of the combination drug. The current pilot study is designed to address the small sample size and provide a proof of concept that LNG plus MEL given 48 hours apart can delay ovulation by ≥7 days compared to placebo of ≤2 days.

A pilot study was performed using MEL 15 mg for 5 days in 3 normal menstruating women with a control no treatment but monitored cycle and a treatment cycle. Treatment was initiated when the participant was within one day of anticipated LH surge based on the control cycle. Using the serum LH surge as the anchor 2 of 3 participants had a delay of three days in the rise of serum progesterone, and there was no effect on the LH surge dynamics.

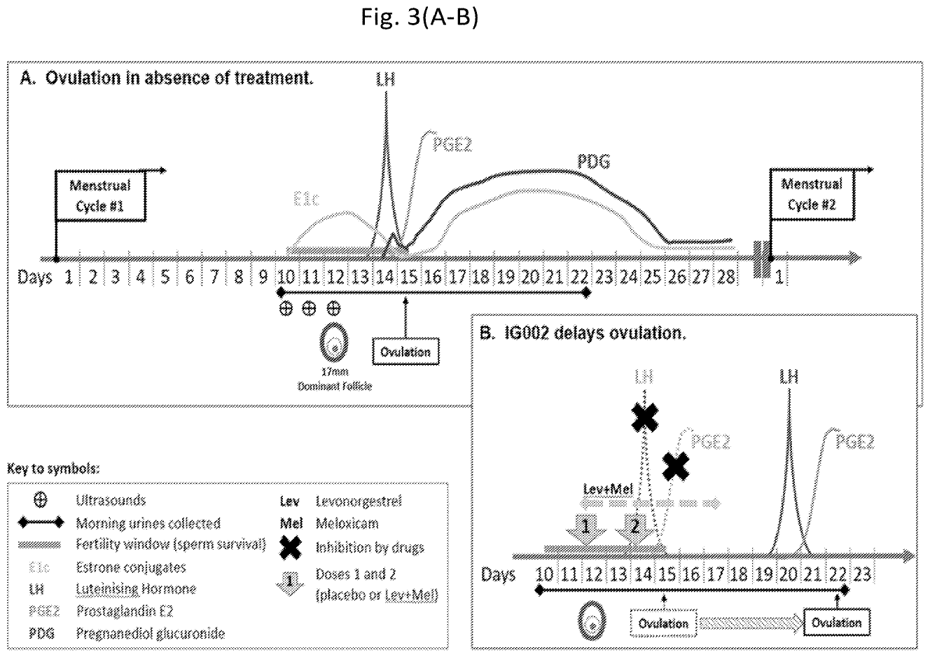

In light of this pilot study and available pharmacokinetic data on two sequential doses of LNG in women, a Proof of Concept study will demonstrate that a second dose of LNG plus MEL taken 48 hours after the first dose of LNG plus MEL will provide an extended delay in ovulation of at least 7 days in normal women compared to placebo. This is accomplished by affecting both the LH surge and intrafollicular cyclooxygenase-2 thus altering two major pathways involved in follicle rupture or ovulation (FIGS. 3A and 3B).

### Example 4: Combination Therapy for on Demand Oral Contraception.

No highly effective on demand oral contraceptive option exists for women. An unmet need for a female-controlled discreet oral contraceptive method taken around the time of intercourse (On Demand) has been identified by women globally (Raymond et al., Contraception, (2014), 90:105-10). Modern effective continuous hormonal contraceptive methods that inhibit ovulation are successfully used by many women, however, the discontinuation rate is very high for these methods. For example 38% discontinue daily oral hormonal contraceptives by 12 months, principally due to unacceptable side effects (Daniels et al., National health statistics reports, (2013), 1-15). Many couples do not need, want to use or can afford daily hormonal or long acting methods such as implants or intrauterine devices/systems (Moreau et al., Contraception, (2007), 76:267-72). Women also frequently change their contraceptive methods, with gaps in the use of highly effective continuous hormonal methods. These gaps are filled by condoms, spermicides, diaphragms and withdrawal methods that are less effective and not discreet (Daniels et al., National health statistics reports, (2013), 1-15; Trussell J., Contraception, (2004), 70:89-96; Duffy et al., 2nd ed: Elsevier, Inc., (2018), pg. 255-60; Simmons et al., American journal of obstetrics and gynecology, (2019), 220:376 e1-e12). Moreover, a woman's contraceptive preference can change as a result of changes in lifestyle, relationships or side effects (Laumann EO, Chicago: University of Chicago Press, (1994), xxxii, 718 p; Venners et al., Hum Reprod, (2006), 21:2272-80). Infrequent intercourse due to the woman's personal life style or changes in relationships is common (Duffy et al., 2nd ed: Elsevier, Inc., (2018), pg. 255-60; Bradley et al., Studies in family planning 2019; 50:3-24). A large Chinese prospective study of working women between ages 21 to 34 years who were married and not using contraception in order to achieve pregnancy; found a range of coital activity between 1 to 6 acts per cycle (month) (Pace et al., Health affairs, (2016), 35:1616-24). Thirty percent of sexually active women in the US have intercourse less than 3 times a year with another 30% reporting intercourse less than 3 times per month (Bradley et al., Studies in family planning 2019; 50:3-24). These data indicate the real-world frequency of female sexual activity. Such women are less likely to use a continuous hormonal contraceptive but in using other less effective contraceptives they are at increased risk of pregnancy. The issues outlined above,

TABLE 1
The InnovaGyn On-Demand female contraceptive will fill a critical gap in available contraceptives

| | | | Pregnancies/100 |
|---|---|---|---|
| Type of Contraceptive | Examples | Major disadvantages | woman/year |
| Long acting; reversible of | IUD; Hormonal Implant | Some methods irreversible; side effects of | <1 |
| irreversible | Surgical Sterilization | long-term hormone therapy | |
| Continuous dosing | Hormonal: Pill, Patch, | Some require regular use (pill); side effects of | 6-9 |
| | Injectable, Vaginal Ring | long-term hormone therapy | |
| Use during sexual | Male/Female Barriers, | Must be used properly at each act of | 12-28 |
| intercourse | Diaphragm, Withdrawal, | intercourse | |
| | Spermicide | | |
| Emergency contraceptive | Hormonal: Plan B, Ella | Increased risk of pregnancy with subsequent | NA; emergency |
| | | act of intercourse or delay between | use only |
| | | intercourse and drug administration | |
| Levonorgestrel 1.5 mg used | Hormonal: Plan B | Not effective if taken after the luteinizing | 11.7 |
| alone with infrequent | | hormone surge begins | |
| intercourse | | | |
| On Demand contraceptive | Innovagyn IG002 | | Goal: <7 |

in the absence of an effective On Demand contraceptive option contribute to a higher risk of unintended pregnancies (Laumann EO, Chicago: University of Chicago Press, (1994), xxxii, 718 p). An effective, discreet and oral contraceptive method taken immediately before or after intercourse would be a significant advance for women with infrequent intercourse and those unable or unwilling to use a continuous hormonal method (Raymond et al., Contraception, (2014), 90:105-10). InnovaGyn, Inc. plans to develop IG002, a combination of levonorgestrel plus meloxicam as a novel On Demand female oral contraceptive with an anticipated Pearl Index (Pregnancy rate) of ≤7 (Table 1).

There are limited studies of Levonorgestrel (LNG)+/−Meloxicam (MEL) taken during the female window of fertility in delaying ovulation. Female fertility is dependent upon ovulation, which is ovarian follicle rupture followed by a release of the oocyte (egg). The midcycle surge of pituitary luteinizing hormone (LH) initiates a series of events within the ovarian follicle culminating in ovulation (Duffy et al., Endocrine reviews, (2019), 40:369-416). The absence of an LH surge results in follicular atresia and anovulation (Croxatto et al., Contraception, (2004), 70:442-50). A single dose of LNG taken prior to the LH surge results in both a delay and a reduction in the peak serum LH levels for 2 days with a subsequent 5-day delay in ovulation (Croxatto et al., Contraception 2001; 63:111-21; Devoto et al., Fertility and sterility, (2005), 84:46-51; Brache et al., Contraception, (2013), 88:611-8). We are aware of only one clinical study which directly compared LNG plus MEL to LNG plus placebo; the endpoint being percentage of follicles with no rupture within 5 days of a single dose of either treatment (Duffy et al., Human reproduction update, (2015). This pilot study reported a single dose of LNG plus MEL given when the maximum follicle diameter was 17 mm delayed ovulation for 5 days in 70% of women but was not significantly different than LNG alone (50%) due to the small sample size of 10 women. The same study gave LNG plus MEL to 31 women with a follicle diameter of ≥18 mm and follicle rupture was delayed for 5 days in 39% of women receiving LNG plus MEL vs 16% of women receiving LNG plus placebo (p=0.052) (Duffy et al., Human reproduction update, (2015). This study is the only clinical study of LNG plus MEL in normal women and the interpretation of their results is hampered by small sample sizes and use of a single dose of the combination drug. We have designed the current pilot study to address the small sample size and provide proof of concept that two doses of LNG plus MEL 48 hours apart can delay ovulation by ≥7 days compared to placebo of ≤2 days. Sexually active women have used LNG alone with intercourse (Glasier et al., Contraception, (2011), 84:363-7). However, LNG used alone with infrequent intercourse by women reporting six or fewer acts of intercourse per month reported a Pearl Index of 11.5 pregnancies per 100 women years of use (Glasier et al., Contraception, (2013), 87:309-13). This was marginally better than barrier methods (e.g. condom, diaphragm) or withdrawal, but not as good as the daily hormonal “Pill” (Table 1) (Gemzell-Danielsson et al., Contraception (2013), 87:300-8). A Cochrane review of LNG alone with intercourse described several small and poorly designed clinical trials with reported Pearl Indices between 5 to 7 per 100 women years of use (Glasier et al., Contraception, (2011), 84:363-7). LNG alone taken when the ovarian follicle reached a mean diameter of 18 mm had limited to no effect on delaying follicle rupture compared to placebo (Brache et al., Contraception, (2013), 88:611-8). This result could have been due to the administration of LNG after the onset of the pituitary LH surge that results in follicle rupture. LNG inhibits, blunts or obtunds the LH surge when taken before the surge begins, but has no effect on LH dynamics after the onset of the surge (Croxatto et al., Contraception 2001; 63:111-21; Brache et al., Contraception, (2013), 88:611-8).

The LH surge also up regulates intrafollicular cyclooxygenase-2 enzyme that is involved in the synthesis of prostaglandin E-2, a critical pathway involved in follicle rupture and ovulation in primates (Halpern et al., Cochrane Database Syst Rev, (2014), 9:CD007595). MEL is a known cyclooxygenase inhibitor that delays and inhibits follicle rupture in primates (Halpern et al., Cochrane Database Syst Rev, (2014), 9:CD007595). MEL alone given to monkeys for 5 days in a model of emergency contraception was highly effective but when taken daily with daily intercourse was ineffective in preventing pregnancy (Duffy et al., Endocrine reviews, (2019), 40:369-416; Festin et al., Hum Reprod (2016). MEL alone administration to normal women for 5 days with an 18 mm follicle delayed follicle rupture based on ultrasound by 5 days (Trussell J., Contraception, (2011), 83:397-404; Jesam et al., Hum Reprod, (2010), 25:368-73). These data support our Hypothesis that a LNG plus MEL combination will increase the ovulatory delay providing a more effective On Demand Female Oral Contraceptive.

The InnovaGyn solution to an effective On Demand contraceptive. We will demonstrate in a study that a second dose of LNG plus MEL taken 48 hours after the first dose of LNG plus MEL will provide an extended delay in ovulation of at least 7 days in normal women compared to placebo. Ovulation delay is accomplished by affecting both the LH surge and intrafollicular cyclooxygenase-2 thus altering two major pathways involved in follicle rupture or ovulation (FIG. 3B).

On Demand should not be confused with emergency contraception. Emergency contraception is an after-the-fact method taken within 72 (LNG 1.5 mg) to 96 (ulipristal acetate 30 mg) hours following intercourse, and only administered once in the menstrual cycle. After using emergency contraception, women are counseled not to have intercourse until their next menstrual period in order to reduce the risk of pregnancy occurring with subsequent sexual acts (Massai et al., Hum Reprod, (2007), 22:434-9). Emergency contraception has a variable effect on preventing pregnancy (50 to 95%) and evidence that ulipristal acetate is superior to LNG in preventing pregnancy as the interval from unprotected intercourse to use increases (Harris et al., Journal of biomedical informatics, (2009), 42:377-81; Santoro et al., American journal of physiology Endocrinology and metabolism, (2003), 284: E521-30). Despite use of emergency contraception, pregnancy risks are increased when taken on the estimated day of ovulation, in obese women, and with subsequent acts of intercourse in the same cycle without contraception (Massai et al., Hum Reprod, (2007), 22:434-9).

Market potential and commercialization path: Of the 61 million American women of childbearing age (15-44 years), 43 million (70%) are at risk of unintended pregnancy of whom 4.4 million (10%) are not using any contraceptive method. The target population for our combination product IG002 consisting of LNG plus MEL is sexually experienced women not using continuous hormonal contraception with infrequent intercourse (defined as less than six times per month) who want to be prepared for planned or unplanned intercourse by having available an effective On-Demand oral contraceptive. Our intention is that these women would take one combination tablet in the 12 to 24-hour period prior to anticipated intercourse (i.e. On-Demand) and a second tablet 48 h later. The expectation of our clinical Proof of Concept study is that this 2-dose protocol taken during the window of fertility will delay ovulation for a minimum of 7 days, thus providing evidence that there will be a significant reduction in the occurrence of unintended pregnancy.

Following a successful clinical study, we will further establish the efficacy and safety of IG002. Those Phase 2 clinical trials will include an extended release, single tablet formulation to be used by obese or non-obese women in the “infrequent intercourse” category. We next plan a Phase 2 clinical study in women at risk of pregnancy who self-describe as having infrequent intercourse and do not want or wish to use current hormonal or barrier contraceptive methods. Phase 3 trials will confirm reduced incidence of unintended pregnancy as a primary endpoint.

There has not been a new or innovative approach to female contraception since “The Pill” in 1960. That contraceptive is required to be taken daily, irrespective of the frequency of intercourse the woman experiences or wants. Being a hormonal-based medication, “The Pill” thus exposes a significant proportion of women who have infrequent intercourse to an unnecessary level of hormonal intake versus the intended outcome: reduced risk of pregnancy. IG002 addresses a major unmet need for an effective, discreet and safe On Demand female-controlled oral contraceptive method for women who do not wish to be exposed to continuous hormones. In addition, IG002 is discreet and does not interfere with the sex act as do barrier contraceptives. The combination of these parameters are wholly consistent with providing women who do not have frequent intercourse a contraceptive giving them control over the timing of taking the medication in advance of the act of intercourse; the frequency with which they need to take the contraceptive; reducing their exposure to hormonal medication; and still having a contraceptive giving a strong reduction in the risk of pregnancy.

Furthermore, dependent on FDA approval, there is potential that this new On Demand contraceptive could be available Over-the-Counter due to the specific nature of the component drugs. These features together would be a major shift in the current clinical paradigm for contraceptive options for women who have infrequent intercourse or who dislike or do not have confidence in barrier or withdrawal methods of contraception.

IG002 is innovative and will be the first new product specifically aimed at inhibiting two major pathways involved in ovulation to effectively prevent pregnancy when used On Demand. IG002 combines inhibition of the pituitary LH surge and intrafollicular PGE2 synthesis, each of which are critical to the process of ovulation. IG002 provides a single dose contraceptive method to be used with infrequent or unexpected acts of intercourse, and thereby provide a reduction of risk of pregnancy closer to that of daily hormonal contraceptive use but without the requirement to take unnecessarily a daily hormonal medication.

IG002 provides an innovative approach for female contraception through a novel combination of two drugs and a novel dosing regimen which together provide sufficient protection (minimum 7 days) to allow for infrequent acts of intercourse in a menstrual cycle and reduced common side effects through reduced hormonal exposure. We anticipate in these studies finding that the additional of meloxicam reduces menstrual/abdominal cramping, unscheduled endometrial bleeding, normal and heavy menstrual bleeding compared to no treatment.

This Phase I study is a single site, prospective, non-randomized, single blind study in normal menstruating women between 18 and 40 years of age who are not at risk of pregnancy, with the Specific Aims of (1) to determine if two doses of a combination of LNG plus MEL, given 48 hours apart, significantly prolong the time to ovulation by at least 7 days in normal women compared to two doses of placebo, also given 48 hours apart; and (2) to determine the occurrence of side effects following two doses of LNG plus MEL compared to two doses of placebo. We have determined that the optimal study is to recruit a cohort of 22 women of normal BMI who will be followed through two consecutive menstrual cycles. This number of participants allows us to conduct a study with scientific rigor and clinically meaningful end points. Twenty-two participants provide sufficient power to detect a significant difference in delay of ovulation between placebo and LNG plus MEL while remaining within the time and cost constraints of a Phase 1 study. Women only will be recruited since this is to be a female contraceptive. Each woman will receive two doses of placebo in the first menstrual cycle; in the second menstrual cycle, each woman will receive two doses of LNG plus MEL. This non-randomized study design avoids the need to extend the study in a randomized design to include a washout menstrual cycle and avoid carry-over effects of LNG plus MEL on the ovarian/menstrual cycle if women were then to receive the placebo doses. Each woman will thus be evaluated for time to ovulation over two consecutive menstrual cycles in which they receive two doses of placebo in the first cycle followed by two doses of LNG plus MEL in the second cycle. The first dose of placebo or LNG plus MEL combination will be taken when the dominant follicle has reached a diameter of 17 mm as determined by ultrasound in each of the two menstrual cycles. By this means we can ensure that the first dose of placebo or LNG plus MEL combination is given at: (A) a consistent stage of dominant follicle development prior to ovulation, the anchor for the outcomes and (B) at a time likely to be ahead of, but close to, the LH surge which would initiate the events leading to ovulation. With this design, we expect to have an optimal strategy to compare the effect of the LNG plus MEL combination vs placebo on the time to ovulation. The options to determine the time of ovulation were two-fold: (A) use ultrasound images to determine the time of ovulation; or (B) measure the urinary metabolites of estrogen (estrone-3-glucuronide) and progesterone (pregnanediol-3-glucuronide) and use the published algorithm method to determine the beginning of corpus luteum formation and thus the time of ovulation. Each method of determining ovulation has pros and cons and each has advocates and protagonists; but each method also has clinically-demonstrable value. We will measure daily urine samples for the two metabolites mentioned above. The daily urine samples will be collected and stored at home by the participants using a standard clinical protocol.

Our study design addresses issues of rigor and reproducibility by: (1) using two doses of the combination of LNG plus MEL spaced 48 hours apart. (2) giving the first dose of LNG plus MEL when the follicle size is 17 mm (just prior to the start of the LH surge); (3) powering the study (n=22) for a significant difference between LNG plus MEL and placebo treatments; and (4) providing the first dose of placebo and drug at the same time in each menstrual cycle using ultrasound measurement of follicle diameter of 17 mm as the anchor for the outcomes.

Experimental Design for each Specific Aim:

Specific Aim #1: Determine the interval from first dose of levonorgestrel plus meloxicam to the luteal-follicular shift in normal women following two doses of active medication compared to two doses of placebo.

There will be a delay of >7 days between the first dose of combination drug and the occurrence of ovulation compared to <3 days following placebo.

Study Design. The study design is an open label non-randomized single blind clinical trial in which each participant will receive two doses of placebo 48 hours apart in their first menstrual cycle and then two doses of LNG plus MEL 48 hours apart in their subsequent menstrual cycle. An overview of the study design is shown in FIG. 1. We will compare the interval from taking the first dose of medication to the development of a functioning corpus luteum based on the shift in the ratio of urinary estrone-3-glucuronide (EC)/pregnanediol-3-glucuronide (PDG). An “n” of 22 was calculated using the Log-Rank Test method and setting the Power to 80% and alpha to 0.05 to test the hypothesis that there was no difference between the “survival curves” for the placebo and drug-treated cycles.

Methods: We will recruit 26 to complete 22 normal menstruating women between 18 and 40 years of age in good general health with a body mass index ≤30, who are using non-hormonal contraceptive methods, abstinence, in an ongoing same-sex relationships or they and their partner have a permanent form of contraception with interruption of the fallopian tubes or vas deferens. To be enrolled, the participant must sign the Informed Consent and have no contraindication to use of levonorgestrel or meloxicam (See Protection of Human Subjects Introduction for specifics)

First morning voided urine will be collected by each participant beginning on menstrual days 10±1.0, and then daily for 12 days. A 3 mL aliquot of the urine will be placed in a storage tube containing glycerol capped and kept in the freezer until returned to the Research Center. Each container will be labeled with the participant initials, participant order number, the cycle number (1 or 2), visit day of collection and the calendar date [Example JAD 001 V1 Day 11). All samples will be stored frozen at −20° C., batched and shipped to the Core laboratory for analysis. Each urine sample will be analyzed for estrone conjugates, luteinizing hormone, and pregnanediol-3-glucuronide using published methods³¹.

All ultrasound and laboratory results will be entered into Redcap data base by the clinical coordinator. All data analysis will be performed by the Statistician using the entered de-identified results. The participant will be seen after the last treatment following a menstrual bleed for a brief medical history with review of bleeding and adverse events from her daily diary card, and have a urine pregnancy test performed. She will be counseled regarding ongoing contraception if indicated and exited from the study.

Expected Outcomes and Data Analysis: We expect that there will be no delay between first dose of placebo and evidence of luteal formation <3 days later using the luteal-follicular transition based on urine EC and PDG ratio and levels. The treatment cycle with two active doses of LNG plus MEL will demonstrate a delay of ≥7 days from first dose to the outcome parameters.

Statistical Analysis: We expect that two doses of a LNG pus MEL combination, dosed 48 h apart with the first dose administered when the dominant follicle has reached 17±1.0 mm in size, will result in the “Day of Luteal-follicular Transition” (DALT) being delayed by at least 7 days vs the time to DALT when a woman received two doses of placebo. The DALT following determination of the ratios of the urinary metabolites of estrone conjugates and progesterone will be identified using published algorithms. Survival analysis/LogRank method was applied to calculate the required sample size. The outcome event is the time until ovulation (DALT) and censored observations (subjects for whom ovulation did not occur during the 10-day follow-up time) are probable. The aim is to compare the survival curves for the 2-doses placebo to the 2-doses treatment group using the length of follow-up for each patient and the Log-Rank Test. The Null Hypothesis is that there will be no significant difference between the two survival curves. The sample size is obtained for a power of 80% and a two-sided test size alpha of 5%. We assume that the ovulation rate in the placebo group is approximately 95% by 10 days after the first dose of placebo. If we want to increase the success rate (failure to ovulate) from 5% in the placebo group to 70% in the treatment group by 10 days after the first dose of drug combination, we will need to recruit to complete 22 subjects. We plan to enroll 26 or more participants to complete a minimum of 22 participants in this study.

Specific Aim #2: Determine occurrence of side effects and safety between two doses of placebo compared to two doses of levonorgestrel plus meloxicam.

Overview: For the women in the clinical trial in Specific Aim 1, we will record reported side effects during use of placebo and LNG plus MEL. Our null Hypothesis is: levonorgestrel plus meloxicam compared to placebo will have limited and mild treatment-emergent side effects and no severe adverse events. Each medication has its own adverse or side effect outcomes. LNG is indicated as a contraceptive and MEL is used for relief of arthritis pain. Age and underlying disease in the older woman can contribute to MEL side effects and adverse outcomes. However, both medications have regulatory agency approval and have tolerable side effects in young healthy women. Serious adverse effects with LNG have not been reported. Serious adverse events associated with MEL are rare, usually involve the gastrointestinal tract, cardiovascular system, liver or skin and are described in depth in the Protection of Human Subjects. Due to the limited exposure to each medication and the use of approved doses of each, we do not anticipate having any serious medication-related side effects. Our proposed clinical trial is to administer two doses 48 hours apart of placebo compared to two doses 48 hours apart of LNG 1.5 mg plus MEL 15 mg in the next menstrual cycle. The infrequent dosing should result in minimal to no adverse events in the participants. There may be reduced abdominal/menstrual cramping, and reduction in the amount of menstrual blood loss in the cycle of the combination therapy compared to placebo.

Methods: Side effects and/or symptoms will be captured using a daily menstrual diary card with the participant recording the day/date of placebo or LNG plus MEL and recording any symptoms or adverse events. Safety assessments will use significant changes in medical history, physical findings, vital signs, and urine analysis between the enrollment and exit visits. All severe adverse events will be reported within 72 hours to the Data Safety and Monitoring Committee, the Institutional Review Board and the US FDA. Adverse events, unscheduled bleeding and outcome events such as delay in ovulation will be reported to the Independent Data Safety and Monitoring Board on a regular basis.

Outcomes: We anticipate that MEL will be associated with abdominal cramping or pain with nausea and headache in less than 10% of the participants during treatment. The relatively small sample size and limited exposure to MEL may not result in a significant incidence of vomiting, diarrhea or skin changes as side effects. MEL use could delay the next menstrual bleed by 2 to 4 days. LNG use may result in unscheduled vaginal bleeding or spotting, with a change of ±2 days in the day of onset of the subsequent menstrual bleed in 20% of the participants. Nausea, abdominal pain and headache could occur with a range of 5 to 10% with use of LNG. Adverse symptoms related to the study medications are anticipated to be mild, limited in duration, and temporally related to the ingestion of the study medications but should not result in discontinuation of the medication nor withdrawing from the study. Transvaginal ultrasound could cause mild discomfort but no pain, and urine collection is a noninvasive method for obtaining a biologic specimen.

A project timeline for the clinical study is shown in Table 2. Successful completion of this Phase I project will demonstrate the utility of using a 48-hour interval between two doses of LNG plus MEL and the consequent extension of the interval to the luteal-follicular transition and PDG rise as an index of efficacy. This finding will contribute vital information toward development of IG002 for further clinical studies. This first step will support the development of a single oral tablet containing LNG and MEL to be used with multiple acts of intercourse in a menstrual cycle. This study provides essential data to support future Phase II application of IG002 in a multicenter clinical contraceptive efficacy study in at-risk women.

Future studies will include: 1) PK study of the new, single dose oral formulation of the combination drug, with extended delivery in normal and obese women, 2) a multicenter Phase 2B contraceptive efficacy study of the new formulation in normal and obese women at risk of pregnancy, 3) Phase III clinical safety and efficacy trial, and 4) analysis of stability and purity of the developed drug throughout the intended shelf life under appropriate storage conditions. Successful completion of these studies would support an FDA New Drug Application.

Example 5: A Phase 2 Study of the Efficacy of Levonorgestrel Plus Meloxicam Taken Twice within 48 Hours to Extend the Interval to Ovulation Compared to Placebo in Normal Women.

Determine the interval between the first dose of levonorgestrel plus meloxicam to ovulation using the luteal-follicular transition in normal women compared to placebo. There will be a delay of ≥7 days between the first dose of combination drug and the occurrence of ovulation compared to <3 days following placebo. The outcome will use the well-characterized luteal-follicular transition that reflects ovulation with formation of the corpus luteum using changes in urinary estrone-3-glucuronide and pregnanediol-3-glucuronide. These data will establish that levonorgestrel plus meloxicam given in two doses is highly effective in delaying ovulation in normal women.

Compare the occurrence of side effects and the safety of levonorgestrel plus meloxicam compared to placebo. Levonorgestrel plus meloxicam compared to placebo will have limited and mild treatment-emergent side effects and no severe adverse events. Medication-related side effects such as unscheduled vaginal spotting or bleeding and potential delay of menses will be mild and transient and there will be no severe adverse events and the treatment will be considered safe based on medical history, the physical examination, vital signs and acceptable bleeding profile by the participant.

This Phase 2 clinical protocol is to determine the duration of the ovulatory delay in normal women when two separate doses of levonorgestrel plus meloxicam are taken 48 hours apart when the dominant ovarian antral follicle is 16-18 mm in diameter in the window of fertility.

This Phase 2 study will provide evidence of the efficacy and safety of two doses of IG002 in delaying ovulation. These findings will provide the “GO” signal for further clinical trials designed to advance IG002 for regulatory approval as a novel On Demand female oral contraceptive.

This Phase 2 clinical trial is designed to provide evidence that the administration of levonorgestrel plus meloxicam taken by the woman in two doses 48 hours apart during her window of fertility will delay ovulation compared to a comparable two doses of placebo in a controlled cycle. The secondary endpoints are safety using adverse event reporting, vital signs and incidence of vaginal spotting and/or bleeding and menstrual delay if any with an acceptability questionnaire at exit along with incidence and severity of treatment emergent side effects.

Levonorgestrel is available in combination hormonal oral contraceptives, a progestin only oral pill, subcutaneous implant, emergency contraceptive pill and a medicated intrauterine device. Levonorgestrel has a long history as a safe and effective progestin without significant side effects. Common side effects are changes in menstrual cycle interval or unscheduled vaginal bleeding and headache, breast tenderness, nausea, abdominal cramps and dizziness. There are no serious side effects reported with the use of levonorgestrel.

Meloxicam is a well-known non-steroidal anti-inflammatory drug (NSAID) used to treat and reduce the pain of arthritis. It is principally taken daily by older men and women for chronic arthritic or muscular pain. There is an increased risk of cardiovascular disease such as deep vein thrombosis and myocardial infarction with chronic daily administration of non-steroidal anti-inflammatory drugs that is also age related. Gastric ulcers and gastrointestinal bleeding are associated with chronic administration of a NSAID at any age. Skin reactions are infrequent but can be life threatening with Stevens Johnson Syndrome and Toxic epidermolysis rare but fatal side effects. Common side effects occurring in more than 5% of daily users in clinical trials are diarrhea, upper respiratory tract infection, dyspepsia and influenza like symptoms.

The combination of levonorgestrel plus meloxicam is being developed as an effective oral female contraceptive taken with intercourse in women who have infrequent intercourse or who do not wish to have continuous hormone exposure with an oral pill, implant or intrauterine device. It has the advantage of being used with the sex act as the reminder to take the medication rather than emergency contraception, which is used after the coital act. Women who have infrequent intercourse use as contraceptives with intercourse: male and female condoms, vaginal diaphragms with spermicides, sympto-thermal methods or withdrawal. This combination On Demand Female Oral contraceptive has the following advantages. This is a female controlled method that is discreet and more effective than condoms, diaphragms, spermicides, symptom-thermal methods and withdrawal. The side effects are minimal due to its infrequent use and the major adverse effect would be unscheduled vaginal bleeding or delay in menstruation and gastro intestinal upset. It would be used at the time of intercourse with intercourse being the recall or prompt to take the medication. The combination product will expand the contraceptive efficacy of single dose levonorgestrel as emergency contraceptive to embrace an efficacy when taken before and after the pituitary LH surge begins which is the initiator of ovulatory events.

The potential risks from levonorgestrel are minimal and are related to unscheduled vaginal bleeding and delay of menstrual onset when used. These side effects are within acceptable safety limits. Meloxicam with daily use has increased risk of cardio-vascular, gastro intestinal and skin diseases associated with chronic use and in older women, but this combination would be used infrequently in young healthy women The combination product is designed to be used with intercourse and >30% of women in the United States have an intercourse frequency of less than 3 acts per month or year. Thus, the infrequent need for this product should result in a much lower incidence of side effects but improved compliance reducing unintended pregnancies.

This product will not prevent sexually transmitted infections (STI) including the human immunodeficiency virus, (HIV) that causes acquired immunodeficiency syndrome (AIDS). Male condoms are effective in preventing STI and HIV infections and male and/or female condoms may be used by the participants with intercourse to prevent these infections during the study.

Blood sampling can result in a blot clot, infection and pain at the venipuncture site. We are limiting the number of venipunctures instead collecting daily morning urine for analysis of hormone metabolites to reflect pituitary-ovarian function. Collection of urine does not carry any risk to the participant.

Transvaginal ultrasound is used frequently to assess ovarian and uterine status and may cause discomfort but has not been associated with pain or reproductive tract problems with frequent use.

Study Design: We will perform a single site, single blind clinical trial to determine the delay in ovulation following administration of placebo or levonorgestrel plus meloxicam in normal menstruating women aged 18 to 40. There will be a delay of >≥7 days between the first dose of combination drug and the occurrence of ovulation compared to ≤3 days following placebo.

We will screen 26 potential participants to enroll and complete 22. Each participant after signing an Informed Consent and meeting all inclusion and exclusion criteria will begin Pre-Treatment Visits on menstrual day 9 of the subsequent menstrual cycle following a negative urine pregnancy test. The participant will undergo a transvaginal ultrasound on menstrual days 9, 11, 13 and possibly day 14 to determine ovarian follicle diameters in two planes (frontal and sagittal) using transvaginal ultrasound. When the largest follicle diameter is 16-18 mm the participant will be given the assigned intervention. Placebo tablets will be given in two doses 48 hours apart in the 1^{st} control cycle and levonorgestrel 1.5 mg plus meloxicam 15 mg two doses 48 hours apart in the 2^{nd} treatment cycle. Each participant will be asked to collect a first morning voided urine sample beginning on menstrual day 9; she will continue to collect daily morning urines for 10 days after the participant is given the first dose of the assigned intervention. The ovarian follicle diameter of 17 mm occurs approximately in the middle of the woman's window of fertility which is the four days preceding the day of ovulation. We anticipate that ovulation will take place within 72 hours after the first placebo dose in >90% of the participants and will be delayed ≥7 days following the first dose of levonorgestrel 1.5 mg and meloxicam 15 mg orally in ≥85% of the participants.

The primary outcome is the delay in days from the first dose of medication to evidence of ovarian corpus luteum formation resulting after ovulation. The daily urine samples will be assayed for luteinizing hormone, estrone-3-glucuronide, and pregnadiol-3-glucronide by our central laboratory. Changes in the urinary metabolites of estrone (estrone-3-glucuronide) and progesterone (pregnanediol-3-glucuronide) are used to identify the day of the luteal-follicular transition (DALT) indicating ovulation (FIG. 1A). Urine luteinizing hormone will be analyzed on a subset of the daily urine samples to confirm a LH increase or surge in the placebo cycle (Days 12 to 17) and the delay if any of the LH surge in the levonorgestrel plus meloxicam cycle (Days 12 to 19) (FIG. 3A).

Safety parameters consisting of blood pressure and pulse obtained at each visit and the incidence of treatment emergent adverse events and any unscheduled vaginal spotting or bleeding captured by the participant using a daily diary card. The participant will be instructed to write down any symptoms or problem along with medications taken including study drug and other medications. The occurrence, percentage and relationship of minor and moderate adverse events will be noted and categorized using Medical Dictionary for Regulatory Activities (MedRA) adverse event classification for each intervention placebo and medication and listed in all reports and publications. Each participant will have blood pressure and pulse obtained at every visit after seated for 5-10 minutes. Adverse events and any resulting interventions will be assessed at each visit. Mean and standard deviation of all vital signs results and the incidence of adverse events before and after treatment will be compiled and listed in all reports and publications.

We expect that two doses of a levonorgestrel/meloxicam combination 48 h apart with the first dose administered when the dominant follicle has reached 16-18 mm in size, will result in the “Day of Luteal Follicular Transition” (DALT) being delayed by at least 7 days vs the time to DALT when a woman received two doses of placebo ≤3 days. The DALT will be determined using changes in the urinary metabolites of estrogen conjugates and progesterone. Survival analysis/LogRank method was applied to calculate the required sample size. The outcome event is the time until ovulation (DALT) and censored observations (subjects for whom ovulation did not occur during the 10-day follow-up time) are probable. The aim is to compare the survival curves for the placebo group and the 2-doses treatment group using the length of follow-up for each patient and the Log-Rank Test. The Null Hypothesis is that there will be no significant difference between the two survival curves. The sample size is obtained for a power of 80% and a two-sided test size alpha of 5%. We assume that the ovulation rate in the placebo group is approximately 95% by 10 days after the first dose of placebo. If we want to increase the success rate (failure to ovulate) from 5% in the placebo group to 80% in the treatment group by 10 days after the first dose of drug combination, we will need to recruit to complete 22 subjects. We plan to screen 26 or more participants to complete a minimum of 22 participants in this study.

Levonorgestrel plus meloxicam (IG002) and placebo will be administered at the study site by site staff. Since study staff will observe medication ingestion, no issues with compliance are anticipated.

Participants will be recruited at one site in the US. This study will be listed on the website ClinicalTrials.gov. Referrals from previous and ongoing participants in this study and other studies are accepted.

Each study participant will participate for approximately 3 months. Each potential participant will be seen in a screening visit during the 1st menstrual cycle. She will be enrolled if all Inclusion and exclusion criteria are met and she has a negative urine pregnancy test on menstrual day 9 of the next menstrual cycle. Transvaginal ultrasound will be performed on menstrual days 9, 11, 13 and 14 or until the maximum diameter of the dominant follicle is 16-18 mm. The oral placebo will be given when the dominant follicle by transvaginal ultrasound is 16-18 mm in one diameter and repeated 48 hours later. Daily morning urine collections will take place starting cycle day 9 and continue for 10 days after the first dose of the assigned intervention is taken. The second menstrual cycle will consist of the same evaluations and urine collection. Levonorgestrel plus meloxicam will be administered orally when the dominant follicle diameter is 16-18 mm and repeated 48 hours later. An exit visit will take place early after the onset of her next menstrual bleed and include assessment of adverse events and concomitant medications, vital signs and physical examination, a urine pregnancy test, collection and review of diaries and administration of an Acceptability questionnaire. The total duration of the clinical trial is 12 months.

We will screen 26 healthy female subjects in order to achieve our goal of completing 22 participants through both investigative cycles. This requires replacing participants if anyone drops out before completion of the study.

Subjects will not be randomized for this study. This is a single blind clinical study with the participant and the clinical research staff knowledgeable regarding the medication. The clinical laboratory for all analyses is blinded to the medication regimen.

Sex hormones or other forms of hormonal contraception are prohibited during study participation. All concomitant treatments will be recorded on the subject's electronic case report forms (eCRF), including the name of the drug, start and stop dates and reason for use from the time of informed consent signing through recovery period and exit visit. Any other investigational medication should be stopped three months before the start of treatment. Other medications for the treatment of inter-current medical conditions should be permitted and recorded as detailed above.

The study medication consists of levonorgestrel 1.5 mg (Plan B one-step, TEVA Pharmaceuticals, Parsippany, NJ) and meloxicam 15 mg (Mobic, Boehringer Ingelheim Pharmaceuticals, Inc., Ridgefield, CT). Placebo is micro cellulose placed in a gelatin capsule of comparable size and weight to levonorgestrel 1.5 mg and meloxicam 15 mg. Both active medication and placebo will be placed in #3gelatin capsule by a local compounding pharmacy and bottles containing each drug supplied to the clinical research staff appropriately labeled for individual dispensing.

Target Population: Healthy women (ages 18 to 40 years) who meet inclusion criteria specific to this trial will be recruited from the general population and from regular visitors to family planning and other clinics serving reproductive aged women seeking gynecologic care or reproductive health services.

Dosage and Administration: Each participant will be administered placebo in the first investigational cycle when the dominant follicle has a diameter of 16-18 mm and the dose repeated 48 hours later. The encapsulated combination of levonorgestrel plus meloxicam will be administered in the second investigational cycle when the dominant follicle diameter is 16-18 mm and repeated 48 hours later.

Duration of Subject Participation: Each study participant will participate for approximately 3 months. Each potential participant will be seen in a screening visit during the 1st menstrual cycle. She will be enrolled if all Inclusion and exclusion criteria are met and she has a negative urine pregnancy test on menstrual day 9 of the next menstrual cycle. Transvaginal ultrasound will be performed on menstrual days 9, 11, 13 and 14 or until the maximum diameter of the dominant follicle is 16-18 mm. The oral placebo will be given when the dominant follicle by transvaginal ultrasound is 16-18 mm in one diameter and repeated 48 hours later. Daily morning urine collections will take place starting cycle day 9 and continue for 10 days after the first dose of the assigned intervention is taken. The second menstrual cycle will consist of the same evaluations and urine collection. Levonorgestrel plus meloxicam will be administered orally when the dominant follicle diameter is 16-18 mm and repeated 48 hours later. An exit visit will take place early after the onset of her next menstrual bleed and include assessment of adverse events and concomitant medications, vital signs and physical examination, a urine pregnancy test, collection and review of diaries and administration of an Acceptability questionnaire. The total duration of the clinical trial is 12 months.

Investigation Product Accountability: The levonorgestrel plus meloxicam and placebo will be administered under supervision to participants orally at the study site by trained research staff. Site staff will record all investigational drug administration, the lot and batch number of active medication and the day and time of administration on the participant's Drug Accountability log. Master Drug Accountability log will record the lot and batch number, date of dispensation, subject number and number of doses.

Investigation Product Storage: Study medication will be stored at the designated research site in a dry place at room temperature (≥15° C. [59° F.] and <30° C. [86° F.]) under controlled and secured conditions, in a locked storage area away from sunlight. Study drug will be accessible only to the study personnel as designated by the Principal Investigator prior to use for each subject. A temperature log for the investigational product storage area will be maintained for the duration of the study.

Participants must meet the inclusion criteria and have no exclusions to be eligible to enter the active study. Eligible participants will be invited to participate in the study and attend pre-treatment visits starting cycle day 9 after a confirmed negative urine pregnancy test. Subjects will be given instructions on morning voided urine collection; they will begin this collection starting cycle day 9 before they come in to the study site. Pre-treatment visits will consist of follicular monitoring using transvaginal ultrasound and peripheral blood samples on menstrual days 9, 11 and 13, and daily once the dominant follicle reaches 15 mm. Both placebo and levonorgestrel plus meloxicam interventions will be given when the dominant follicle measures 16-18 mm in any diameter. After the first study medication dose, participants will continue to collect their morning voided urine for 10 days and fill out daily diary cards.

Approximately 10 visits after enrollment visit are anticipated for all women in the monitoring and follow up phase of the study. Throughout the study, women will be encouraged to contact their study site and report by phone or visit any unusual event(s) including medical problems. Extra visits will be arranged if indicated. If necessary, the investigator or study site staff member will contact the participant more frequently. (E-mail or text message communication may be used based on subject preference for study appointment reminders to assist with participant compliance with study visit schedule. E-mail may be used for collecting follow-up information based on individual site HIPAA and encryption allowances).

The study will include 2 menstrual cycles which consist of a monitoring phase of follicular development for approximately one week, followed by 10 days of morning voided urine collection at home, and continual diary collection until the onset of the participant's next menses. Participants will return for an exit visit 5 to 10 days after the subsequent onset of menses post treatment. The exit visit will consist of assessment of adverse events and concomitant medications, vital signs and physical examination, a urine pregnancy test, collection and review of diaries and administration of an Acceptability questionnaire.

Laboratory Tests and Clinical Evaluations: Blood samples collected at the Screening Visit for laboratory analyses are a complete blood count, Chemistry panel, urinalysis and progesterone, The enrollment visit and follow up Pre-Treatment visits with transvaginal ultrasounds will collect a peripheral blood sample for estradiol and luteinizing hormone.

The Investigator or their assigned designee must review and sign all laboratory reports and file a copy with each subject's chart. All out-of-range laboratory results must be assessed by the Investigator for clinical significance. Any reports obtained from the screening assessment must be reviewed, signed and dated on or before the date/time of enrollment in order to properly document the determination of eligibility. If any screening chemistry values are greater than 2 times the upper limit of normal (ULN), then the Medical Monitor must review the lab values prior to enrollment even if the value is not part of the Inclusion or Exclusion criteria for the study.

The CBC, blood chemistry samples, lipids, serum hormones and urinalysis will be analyzed at the local certified laboratories. All local laboratory values will be recorded on eCRFs. All urine hormone analysis will be performed at a central Laboratory.

CBC and Clinical Chemistries: will use a local commercial laboratory for CBC, Chem 9, lipids and urinalysis. Peripheral blood samples collected at Screening include complete blood count (CBC) and clinical chemistries. The CBC testing will include platelets and differential (RBC, WBC, Hemoglobin, Hematocrit, % Basophils, % Eosinophils, % Lymphocytes, % Monocytes, % Neutrophils, and Platelets) and clinical chemistries will include basic metabolic panel and lipid profiles (glucose, creatinine, calcium, potassium, sodium, chloride, BUN, total cholesterol, HDL, LDL, and triglycerides).

Hormone Analysis: A peripheral venous blood sample for progesterone will be obtained during the screening visit to assess ovulation. Peripheral blood samples for estradiol and luteinizing hormone levels will be obtained on menstrual days 9, 11, 13 and at each additional pre-treatment visit during the two treatment cycles with analysis at a local laboratory. Daily urine samples from menstrual day 9 through 10 days after the first dose of the assigned intervention is given will be obtained for analysis of estrone-3-glucuronide and pregnanediol-3-glucuronide by the central laboratory.

Urinalysis: Midstream urine is collected for dipstick urinalysis (Screening) and may be sent to the central lab for further analysis in accordance with the Investigator's assessment of the result. Women with abnormal urine dipstick results and/or reports of symptoms indicative of a UTI will have a full urinalysis completed at the local laboratory to determine clinical significance and need for treatment prior to approval for enrollment.

Pregnancy Testing: Urine Pregnancy (uCG) testing is performed as part of screening, on menstrual day 9 for both the placebo and treatment cycle and at the exit visit. Serum hCG testing will be done only if the urine pregnancy test is positive. Pregnancy testing will also be performed whenever there is any question about whether a woman might be pregnant at any time during the study. Confirmatory serum hCG samples will be tested locally for a quantitative hCG.

Pap/STI: At screening, the Investigator will determine if a Pap test is needed. A Pap test will be completed for the potential participant at screening if the potential participant is not in compliance with ASCCP or ACOG Guidelines. If a potential subject states that she has had a Pap test within the compliance requirements, then she will need to provide documentation of the acceptable test results.

Potential participants will be screened for chlamydia, gonorrhea and trichomonas and the sample will be sent to a local lab. Potential participants diagnosed at screening with chlamydia, gonorrhea or trichomonas infection may be included in the trial following treatment completion per local standards. Women with a history of genital herpes can be included if outbreaks are infrequent and deemed appropriate in the opinion of the Principal Investigator or medical designee assessment and local standards of practice. Antiviral prophylactic therapy is permitted. It is advisable for partners of women who screen positive for sexually transmitted infections to be treated as well.

Transvaginal Ultrasound: The participant will undergo a transvaginal ultrasound on menstrual days 9, 11, 13 and possibly day 14 or more to determine ovarian follicle diameters in two planes (frontal and sagittal) using transvaginal ultrasound. When the largest follicle diameter is 16-8 mm the participant will be given the assigned intervention.

Diaries: Daily Diaries will be dispensed at the screening visit and reviewed at the beginning of each cycle until the Exit Visit. Subjects will complete a daily diary of vaginal spotting or bleeding and symptoms (headache, nausea, acne, mood/depression, menstrual pain, or any other notable events). After the return to menses, the characteristics of the menstrual cycle will be recorded (length, intensity, duration of bleeding, symptoms). All original diary cards will be retained at the site.

Medication Acceptability & Satisfaction Questionnaire: A medication acceptability questionnaire developed for this study will be administered for the subjects to answer questions about their acceptability of levonorgestrel plus meloxicam as a product and as a method for contraception. The questionnaire will be administered at the Study Exit Visit.

Safety Assessments: Safety assessments will include:
Adverse event reports
Vital signs and physical exams
Unscheduled vaginal spotting or bleeding
Delay of onset of menses
Pregnancy monitoring

Visit Procedures: There will be a single cohort of women who will complete two sequential menstrual cycles: a placebo and a treatment cycle. Each participant will complete approximately 14 study visits during each menstrual cycle. Additional visits may be necessary to meet intervention criteria, and/or appropriate medical follow-up care if required or depending on the duration of time until the subject returns to normal menses. All participant data will be recorded on source documents. Participant names will not be recorded on the eCRFs; instead, unique subject identifiers will be assigned. Data will be collected during an initial screening visit to assess subjects' eligibility. Eligible participants will continue on to the Pre-Treatment visit, which will occur on menstrual day 9 of the subsequent menstrual cycle. Participants will be considered enrolled after confirmation of a dominant follicle reaching 16-18 mm. Subjects who are anovulatory may repeat the cycle once.

Missed visits or assessments should be completed as soon as possible prior to the next scheduled visit. Subsequent visits should not be rescheduled based on a change in visit days due to the missed/moved visit. If a missed visit or assessment cannot occur prior to the next regularly scheduled visit, the missed visit/assessment will be recorded as such.

Pre-Screening Period: Potential research participants will call the clinical site and one of the research staff will carry out the following procedures over the telephone:
The study will be explained
Verbal consent for taking a screening history will be obtained over the phone
A screening history will be taken
If the potential participant appears eligible by screening history, her mailing address (or email address) will be obtained so that a consent form, history form, and other administrative forms can be sent for review and completion prior to the screening appointment
Screening Visit appointment will be made for menstrual cycle day 20-24

Screening Visit: The Screening visit will be scheduled for menstrual cycle day 20-24. Prior to conducting any screening procedures, potential participants will be given a full description of the nature of the study. The purpose, timing, procedures and risks of the study will be explained to the woman, including requirements for enrollment and participation in the study, medication restrictions during the study, and requirements for washout of certain medications that the woman may already be taking and all of her questions will be addressed. The eligible woman, who is willing to participate in the study will then be required to provide both a verbal and written consent. The Investigator or a designated medically qualified member of the study staff will interview potential participants to establish their eligibility for inclusion in the study. Potential subjects will be screened according to the inclusion and exclusion criteria.

### Pre-Treatment Visits—Menstrual Cycle Days 9-18 and Enrollment Visit

Based on the laboratory results and Inclusion/Exclusion criteria, the Investigator will assess the eligibility of the subject. If the potential participant qualifies, the following procedures will be performed:
Perform in house urine pregnancy test. If the UPT is negative, the participant begins Pre-Treatment procedures. This will only be performed on cycle day 9 of each cycle.
Assess participant for AEs and Concomitant medicines. Review bleeding profile and adverse event diary and collect completed pages. Collect the morning urine sample from the subject for storage.
Obtain sitting blood pressure and pulse
Obtain a peripheral blood sample for estradiol and luteinizing hormone levels.
A transvaginal ultrasound will be performed to measure follicular development and uterine stripe thickness
The participant will be reminded to continue to collect her morning urine
The participant will be scheduled for her next follicle monitoring visit on cycle day 11. Visits will continue on cycle days 11 and 13, and daily once the dominant follicle reaches 15 mm. Once the dominant follicle reaches 16-18 mm in any diameter, the subject will take the assigned intervention (Cycle 1: placebo, Cycle 2: levonorgestrel plus meloxicam).

Dose 1: When the participant has a dominant follicle measuring 16-18 mm in any diameter she will be instructed to take the assigned intervention under supervision (Cycle 1: placebo, Cycle 2: levonorgestrel plus meloxicam). She will be instructed to continue to collect her first voided morning urine for the next 10 days. Schedule the participant to return to the site in 48 hours to receive the second dose of the assigned intervention.

Dose 2 (48 hours after initial dose): The participant will return to the site 48 hours after receiving the initial dose of the assigned intervention (Cycle 1: placebo, Cycle 2: levonorgestrel plus meloxicam). The following procedures will be performed:
Assess participant for AEs and Concomitant medicines
Assess sitting blood pressure and pulse
Medication dispensed and taken under supervision
Collect morning urine samples from subject for storage
Review bleeding profile and adverse event diary and collect completed pages
Remind participant to continue to collect her first voided morning urine
Schedule her to return to the site on approximately cycle day 18 to return urine samples and to monitor for side effects.

Mid-Cycle Urine Collection (approximately cycle day 18): The participant will return to the site on approximately cycle day 18 to return urine samples for processing. The following procedures will be performed:
Assess participant for AEs and Concomitant medications
Assess sitting blood pressure and pulse
Collect morning urine samples from subject for storage
Review bleeding profile and adverse event diary and collect completed pages
Remind participant to continue to collect her first voided morning urine until the assigned date
Schedule her to return to the site approximately cycle day 22 or 6 days after the second treatment dose to return urine samples to the site.

End of Cycle Urine Collection (approximately CD 22): The participant will return to the site on approximately cycle day 22 or 6 days after the second treatment dose to return urine samples for processing.
Assess participant for AEs and Concomitant medications
Assess sitting blood pressure and pulse
Collect morning urine samples from subject for storage
Review bleeding profile and adverse event diary and collect completed pages
Provide collection supplies for morning urine to be obtained on Day 9 of next menstrual cycle.
   She will be scheduled to return to the site on cycle day 9 of her subsequent menstrual period to begin the final cycle of the study. Subject will repeat all visits and procedures as stated in Sections 15.2-15.6 for the levonorgestrel plus meloxicam cycle.

Exit Visit: The participant will return to the site between cycle days 5-10 days of her subsequent menstrual period following the end of the Treatment (levonorgestrel plus meloxicam) cycle (once menses has ceased). The following procedures will take place at the Exit Visit:
Assess participant for AEs and Concomitant medications
Assess sitting blood pressure and pulse
Perform a UPT
Perform a directed physical examination, as needed
Collect all completed diary cards
Collect all remaining morning urine samples from subject for storage
Have participant complete the acceptability questionnaire

### Pregnancy Determination and Follow-Up

The incidence of pregnancy will be assessed. Information on pregnancy will be recorded on all subjects becoming pregnant during their study participation after signing consent through their last follow up contact.

During the course of the study, if a subject is found to be pregnant, the subject must be discontinued from the study and all procedures for the Early Discontinuation/Exit Visit should be performed. The investigator must submit the pregnancy with the EDC within 24 hours of determination and record the pregnancy information in the subjects' medical file. Upon EDC submission, the InnovaGyn, Inc. Medical Monitor will be notified of the pregnancy.

Please note, every effort should be made to ensure the collection of local serum hCG samples and an ultrasound for gestational age when a subject becomes pregnant, which are part of the standard Exit procedures that should be completed.

The estimated date of ovulation will be made by the Investigator based upon results of the following pregnancy determination criteria, listed in order from most accurate to least accurate:
Transvaginal ultrasound.
Quantitative β-hCG determination.
Daily diary information (e.g., last menstrual period).
Estimate based on pelvic and/or abdominal examination or pregnancy outcome.
Investigator estimation in the absence of above criteria.

Subjects who become pregnant during their study participation will be informed/counselled of pregnancy options and referred for appropriate care. All pregnancies will be monitored until outcome (i.e., 9 months follow-up or until outcome information is obtained). Pregnancy outcome data will include the rates of spontaneous abortion, stillbirth and live pre-term and full-term births. Information also will be collected on any maternal or fetal complications. Miscarriages requiring hospitalization, congenital anomalies or birth defects will be classified as serious adverse events (SAEs). Pregnancy outcome data will be collected for analysis. Reports of pregnancy outcome and examinations during the pregnancy will be requested from the outside physician/clinic, if applicable. Every reasonable attempt should be made to obtain outcome data, including hospital and/or physician records.

Pregnancy itself is not an SAE for this study. Pregnancy outcome data will include the rates of spontaneous abortion, stillbirth, live preterm, and full-term births. If a subject has a spontaneous abortion or ectopic pregnancy this will not be considered as an SAE unless it meets one of the SAE criteria indicated in Section 16.4.1 In addition, congenital malformations and anomalies will be recorded and summarized.

If a subject continues with her pregnancy through childbirth, every effort will be made to maintain contact with her so that health of the baby can be evaluated at six and twelve months. A clinical summary of the pregnancy, prenatal and postnatal events will be reported separately from the case report form in narrative format in the clinical summary report. Any outstanding data after the end of the study that is collected will be submitted to the FDA as part of the clinical study report. Pregnancy outcome information will be recorded and the data will be analyzed within the drug safety assessment.

Medical Monitor: InnovaGyn will maintain a Medical Monitor. David F. Archer, MD, is the Medical Monitor for the protocol. Dr. Archer has experience with NIH clinical protocols. He is board certified in Obstetrics & Gynecology with advance certification in reproductive endocrinology and infertility. Dr. Archer will be responsible for evaluating requests for waivers, reviewing protocol safety information and providing the final recommendation based on the input from the DSMB. The Medical Monitor will be tasked to review the adverse events and safety considerations at routine intervals during the study. The safety review by the Medical Monitor will be documented.

### Evaluations

### Pharmacodynamic Parameters

Primary Endpoint: The delay in days from the first dose of medication to evidence of ovulation based on hormonal evidence of ovarian corpus luteum formation. Daily urine samples will be assayed for luteinizing hormone, estrone-3-glucuronide, and pregnadiol-3-glucronide by our central laboratory. Changes in the urinary metabolites of estrone (estrone-3-glucuronide) and progesterone (pregnandiol-3-glucuronide) are used to identify the day of the luteal-follicular transition (DALT) indicating ovulation. Urine luteinizing hormone will be analyzed on a subset of the daily urine samples to confirm a LH increase in the placebo cycle (Days 12 to 17) and any delay in the LH surge in the levonorgestrel plus meloxicam cycle (Days 12 to 19).

Secondary Endpoints: The secondary outcomes are safety parameters consisting of blood pressure and pulse obtained at each visit, the occurrence of any unscheduled vaginal spotting or bleeding and the onset of each menstrual bleed plus the number of bleeding days and the incidence of treatment emergent adverse events captured by the participant using a daily diary card. The participant will be instructed to write down any symptoms or problem along with medications taken including study drug and other medications. The occurrence, percentage and relationship of minor and moderate adverse events will be noted and categorized using Medical Dictionary for Regulatory Activities (MedRA) adverse event classification for each intervention placebo and medication and listed in all reports and publications. Each participant will be involved for a study period of approximately 3 months. Each participant will undergo a complete history and physical evaluation at entry and a brief interim history and vital signs at exit with height and weight at entry. Mean and standard deviation of all vital signs results and the incidence of adverse events before and after treatment will be compiled and listed in all reports and publications.

### Safety Evaluations

Each participant's health status will be monitored carefully throughout the trial. The Medical Monitor will closely monitor all AEs and will inform the site with pertinent changes to safety risks throughout the study. Safety parameters include enumeration of all adverse events (AE) and vital sign monitoring at each visit. Pregnancy testing will be conducted every at screening, prior to treatment or anytime the subject is concerned about pregnancy. Subjects will be questioned at each contact about how they have been feeling and any illnesses they have experienced. Interviews with subjects will include a review of any AE reported in order to obtain as much information as possible. The AEs will be recorded at each visit on the CRF and on the appropriate forms. The principal investigator (or designated medical designee) will review each study event, assess causality and report it in accordance with established procedures. There will be strict adherence to SAE reporting (see Section 16). The IRB will be notified of any serious, unexpected adverse events in compliance with IRB research reporting requirements. The study sponsor will submit expedited safety reports as required to FDA.

This is not a contraceptive study; therefore, subjects will be required to use a non-hormonal method, be surgically sterile (or have a partner that is vasectomized) or be heterosexually abstinent during the study. In the event of any contraceptive failures, pregnancy outcomes will be monitored, recorded on appropriate forms and reviewed.

### Statistical Methods and Considerations

Sample Size Consideration We expect that two doses of a levonorgestrel plus meloxicam combination, dosed 48 h apart with the first dose administered when the dominant follicle has reached 16-18 mm in size, will result in the “Day of Luteal-follicular Transition” (DALT) being delayed by at least 7 days vs the time to DALT ≤3 days when a woman received two doses of placebo. The DALT following determination of the ratios of the urinary metabolites of estrone conjugates and progesterone will be identified using published algorithms (Santoro et al., American journal of physiology Endocrinology and metabolism, (2003), 284: E521-30). Survival analysis/LogRank method was applied to calculate the required sample size. The outcome event is the time until ovulation (DALT) and censored observations (subjects for whom ovulation did not occur during the 10-day follow-up time) are probable. The aim is to compare the survival curves for the 2-doses placebo to the 2-doses treatment group using the length of follow-up for each patient and the Log-Rank Test. The Null Hypothesis is that there will be no significant difference between the two survival curves. The sample size is obtained for a power of 80% and a two-sided test size alpha of 5%. We assume that the ovulation rate in the placebo group is approximately 95% by 10 days after the first dose of placebo. If we want to increase the success rate (failure to ovulate) from 5% in the placebo group to 80% in the treatment group by 10 days after the first dose of drug combination, we will need to recruit to complete 22 subjects. We plan to screen 26 or more participants to complete a minimum of 22 participants in this study.

### Analysis Populations:

Only women will be enrolled in this study since this is a female contraceptive. Women between 18 to 40 years of age with regular menstrual intervals and in good health by history and physical examination will be enrolled in the clinical study.

### Disposition of Subjects

The number of subjects, who are enrolled, treated, attended the various assessments and who complete the clinical trial will be tabulated.

### Primary Analysis

The delay in days from the first dose of medication to evidence of ovulation based on hormonal evidence of ovarian corpus luteum formation. Daily urine samples will be assayed for luteinizing hormone, estrone-3-glucuronide, and pregnadiol-3-glucronide by our central laboratory. Changes in the urinary metabolites of estrone (estrone-3-glucuronide) and progesterone (pregnandiol-3-glucuronide) are used to identify the day of the luteal-follicular transition (DALT) indicating ovulation. Urine luteinizing hormone will be analyzed on a subset of the daily urine samples to confirm a LH increase in the placebo cycle (Days 12 to 17) and any delay in the LH surge in the levonorgestrel plus meloxicam cycle (Days 12 to 19).

### Secondary Analysis

Safety parameters consisting of blood pressure and pulse obtained at each visit, the occurrence and duration of vaginal spotting or bleeding and the incidence of treatment emergent adverse events captured by the participant using a daily diary card. The participant will be instructed to write down any symptoms or problem along with medications taken including study drug and other medications. The occurrence, percentage and relationship of minor and moderate adverse events will be noted and categorized using Medical Dictionary for Regulatory Activities (MedRA) adverse event classification for each intervention placebo and medication and listed in all reports and publications.

### Bleeding Profile

The number of bleeding or spotting days will be summarized through the study and up to the return of menses post treatment. If a subject does not have bleeding and spotting data on all days within an interval, then the missing data will be imputed. The missing data for a subject will be replaced with the average number of bleeding or spotting days recorded in their diary during that interval. If the subject is missing data on >25% of the days within the interval being summarized, then the subject will be excluded from the summary for that interval. Time to menses after the study treatment will be calculated and summarized descriptively. Menses will be defined as the first bleeding that occurs after the intervention is given. After the return to menses, the characteristics of the menstrual cycle will be recorded (length, intensity, duration of bleeding, and any menstrual symptoms).

### Enrollment Plan

We will screen 26 healthy female subjects in order to achieve our goal of completing 22 participants through both investigative cycles. The subjects will be reflective of the demographics of eastern North Carolina and will include participants of white, black, Asian, non-Hispanic white, Hispanic, Native American, Pacific Islander, Alaskan and other races.

The study team will try to retain all subjects and subjects who drop out or are discontinued from the study may be replaced if additional individuals are needed for any dose/route of delivery/BMI group.

### On Demand Visit Flow Chart

| | | Control | | | | Treatment | | | | |
|---|---|---|---|---|---|---|---|---|---|---|
| | | Cycle | | | End of | Cycle | | | End of | |
| | | Pre- | Control | Control | Cycle | Pre- | Treatment | Treatment | Cycle | |
| | | Treatment | Cycle | Cycle | Urine | Treatment | Cycle 2 | Cycle 2 | Urine | EXIT |
| Procedures | Screen^{a} | Visits^{b} | DOSE 1^{c} | DOSE 2^{d} | Collection | Visits^{b} | DOSE 1^{c} | DOSE 2^{d} | Collection | visit^{H} |
| ICF | X | | | | | | | | | |
| Med HX | X | | | | | | | | | X |
| GI Ulcer | X | | | | | | | | | |
| Screening | | | | | | | | | | |
| Con Med | X | X | | X | X | X | | X | X | X |
| AEs | | X | | X | X | X | | X | X | X |
| Vitals^{e} | X | X | | X | X | X | | X | X | X |
| Height and | X | | | | | | | | X | |
| Weight | | | | | | | | | | |
| CBC | X | | | | | | | | | |
| Chemistries | X | | | | | | | | | |
| Lipids | X | | | | | | | | | |
| Urine | X | | | | | | | | | |
| Dipstick/ | | | | | | | | | | |
| Urinalysis | | | | | | | | | | |
| Pap Test | X | | | | | | | | | |
| P4 | X | | | | | | | | | |
| Estradiol | | X | | | | X | | | | |
| Luteinizing | | X | | | | X | | | | |
| hormone | | | | | | | | | | |
| Physical | X | | | | | | | | | X |
| Examination | | | | | | | | | | |
| UPT | X | X | | | | X | | | | X |
| TVU | | X | | | | X | | | | |
| Medication | | | X | X | | | X | X | | |
| Dispensing | | | | | | | | | | |
| Daily Urine | | X | | X | X | X | | X | X | X |
| Collection^{f} | | | | | | | | | | |
| Dispense Diary | X | X | | X | X | X | | X | X | X |
| and | | | | | | | | | | |
| Collect^{G} | | | | | | | | | | |

^{a}Screening to occur on CD 20-24
^{b}Pretreatment visits will start on CD 9. Frequency and number of visits is dependent on follicle monitoring and up to the discretion of the PI. Each visit has a blood draw for estradiol and luteinizing hormone and a transvaginal ultrasound for uterine stripe and ovarian follicle size.
^{c}Initial dose will occur when dominant follicle measures 16-18 mm in the largest diameter and after all pre-treatment visit procedures are completed
^{d}Second dose to occur 48 hours after initial dose
^{e}Vitals consist of sitting blood pressure and pulse
^{f}Collect daily first void morning urine beginning cycle day 9 and ending 10 days after first dose. Aliquot urine into supplied capped tube and refrigerate.
^{G}Dispense daily diaries at screening Visit and collect once cycles are complete
^{H}Exit visit between cycle day 5 to 10 after treatment cycle 2 menstrual bleed.

While there have been shown and described what are presently believed to be the preferred embodiments of the present invention, those skilled in the art will realize that other and further embodiments can be made without departing from the spirit and scope of the invention described in this application, and this application includes all such modifications that are within the intended scope of the claims set forth herein. All patents and publications mentioned and/or cited herein are incorporated by reference to the same extent as if each individual publication was specifically and individually indicated as having been incorporated by reference in its entirety.

1. A method of contraception in a female subject at risk of initiating an unwanted pregnancy, comprising administering to the female subject an effective amount of a combination of a progesterone receptor agonist and a cyclooxygenase-2 inhibitor in each of two doses, wherein the first dose is administered within about 24 hours before or after a first act of sexual intercourse, and wherein the second dose is administered between about 36 hours and about 60 hours after the first dose.

2. The method of claim 1, wherein the second dose is taken within about 12 hours before or after a second act of sexual intercourse.

3. The method of claim 1, wherein the first dose is taken within 24 hours before the first act of sexual intercourse.

4. The method of claim 1, wherein the first dose is taken within 24 hours after the first act of sexual intercourse.

5. The method of claim 1, further comprising administering to the female subject at least one additional dose of said combination after administration of the second dose.

6. The method of claim 1, wherein the progesterone receptor agonist is levonorgestrel and the cyclooxygenase-2 inhibitor is meloxicam.

7. The method of claim 1, wherein the female subject has three or fewer acts of sexual intercourse within any one week of her menstrual cycle and six or fewer acts of sexual intercourse within her menstrual cycle.

8. The method of claim 1, wherein each dose includes from about 0.75 mg to about 3.0 mg of levonorgestrel and from about 7.5 mg to about 30 mg of meloxicam.

9. The method of claim 1, wherein each dose is administered orally.

10. The method of claim 1, wherein each administered dose is in the form of a slow release oral tablet.

11. The method of claim 10, wherein the tablet for the first dose is identical to the tablet for the second dose.

12. The method of claim 10, wherein the slow release oral tablet is formulated to include one or more polymers or waxes.

13. The method of claim 11, wherein the polymer(s) include biodegradable polyorthoesters or polyanhydrides.

14. The method of claim 1, wherein the doses are administered buccally, parenterally, transdermally, vaginally, or via a uterine route.

15. The method of claim 11, wherein the progesterone receptor agonist is levonorgestrel and the cyclooxygenase-2 inhibitor is selected from the group consisting of indomethacin, diclofenac, naproxen, ibuprofen, nimesulide, ketoprofen and piroxicam.
